(19) United States
(12) Patent Application Publication
(10) Pub. No.: US 2007/0258900 A1
(43) Pub. Date: Nov. 8, 2007

(54) Blood Flow Assessment of Clostridial Toxin Applications

(76) Inventor: Joseph Francis, Aliso Viejo (CA)

Correspondence Address: ALLERGAN, INC., 2525 DUPONT DRIVE, T2-7H, IRVINE, CA, 92612-1599, US

(21) Appl. No.: 11/767,448

(22) Filed: Jun. 22, 2007

Related U.S. Application Data
(63) Continuation-in-part of application No. 11/571,197.
(60) Provisional application No. 60/651,493, filed on Jul. 20, 2004.
(60) Provisional application No. 60/805,509, filed on Jun. 22, 2006.

(51) Int. Cl. A61K 49/00; A61K 39/08

(52) U.S. Cl. 424/9.1; 424/239.1

(57) ABSTRACT

The present specification relates to methods for assessing the physiological activity of a target site being evaluated for potential administration of a Clostridial toxin, methods for administering a Clostridial toxin to a particular target site, methods for assessing the effect of an administration of a Clostridial toxin in a mammal, methods assessing the extent of dispersal of a Clostridial toxin from a target area to a non-target area in a mammal, methods of identifying a neurogenic inflammation inhibitor, and methods of assessing an inhibitory effect of a Clostridial toxin on neurogenic inflammation in a target site of a mammal.

This application is a continuation-in-part and claims priority pursuant to 35 U.S.C. § 120 to U.S. patent application Ser. No. 11/571,197, filed Dec. 22, 2006, a national stage application under 35 U.S.C. § 371 of PCT application PCT/US2005/026290, filed on Jul. 20, 2005 which claims priority pursuant to 35 U.S.C. §119(e) to U.S. provisional patent application Ser. No. 60/651,493, filed Jul. 20, 2004, which was converted on Jul. 5, 2005 from U.S. nonprovisional patent application Ser. No. 10/894,851 filed Jul. 20, 2004 and claims priority pursuant to 35 U.S.C. §119(e) to provisional application Ser. No. 60/805,509 filed Jun. 22, 2006, each of which is hereby incorporated by reference in its entirety.

All of the patents and publications cited in this application are each hereby incorporated by reference in its entirety.

The ability of Clostridial toxins, such as, e.g., Botulinum neurotoxins (BoNTs), like, BoNT/A, BoNT/B, BoNT/C1, BoNT/D, BoNT/E, BoNT/F and BoNT/G, and Tetanus neurotoxin (TeNT), to inhibit neuronal transmission are being exploited in a wide variety of therapeutic and cosmetic applications, see e.g., William J. Lipham, COSMETIC AND CLINICAL APPLICATIONS OF BOTULINUM TOXIN (Slack, Inc., 2004). As an example, BOTOX® is currently approved in one or more countries for the following indications: achalasia, adult spasticity, anal fissure, back pain, blepharospasm, bruxism, cervical dystonia, essential tremor, glabellar lines or hyperkinetic facial lines, headache, hemifacial spasm, hyperactivity of bladder, hyperhidrosis, juvenile cerebral palsy, multiple sclerosis, myoclonic disorders, nasal labial lines, spasmodic dysphonia, strabismus and VII nerve disorder. In addition, Clostridial toxins therapies are proposed for treating neuromuscular disorders, see e.g., Kei Roger Aoki et al., Method for Treating Neuromuscular Disorders and Conditions with Botulinum Toxin Types A and B, U.S. Pat. No. 6,872,397 (Mar. 29, 2005); Rhett M. Schiffman, Methods for Treating Uterine Disorders, U.S. Patent Publication No. 2004/0175399 (Sep. 9, 2004); Richard L. Barron, Methods for Treating Ulcers and Gastroesophageal Reflux Disease, U.S. Patent Publication No. 2004/0086531 (May 7, 2004); and Kei Roger Aoki, et al., Method for Treating Dystonia with Botulinum Toxin C to G, U.S. Pat. No. 6,319,505 (Nov. 20, 2001); eye disorders, see e.g., Eric R. First, Methods and Compositions for Treating Eye Disorders, U.S. Patent Publication No. 2004/0234532 (Nov. 25, 2004); Kei Roger Aoki et al., Botulinum Toxin Treatment for Blepharospasm, U.S. Patent Publication No. 2004/0151740 (Aug. 5, 2004); and Kei Roger Aoki et al., Botulinum Toxin Treatment for Strabismus, U.S. Patent Publication No. 2004/0126396 (Jul. 1, 2004); pain, see e.g., Kei Roger Aoki et al., Pain Treatment by Peripheral Administration of a Neurotoxin, U.S. Pat. No. 6,869,610 (Mar. 22, 2005); Stephen Donovan, Clostridial Toxin Derivatives and Methods to Treat Pain, U.S. Pat. No. 6,641,820 (Nov. 4, 2003); Kei Roger Aoki, et al., Method for Treating Pain by Peripheral Administration of a Neurotoxin, U.S. Pat. No. 6,464,986 (Oct. 15, 2002); Kei Roger Aoki and Minglei Cui, Methods for Treating Pain, U.S. Pat. No. 6,113,915 (Sep. 5, 2000); Martin A. Voet, Methods for Treating Fibromyalgia, U.S. Pat. No. 6,623,742 (Sep. 23, 2003); Martin A. Voet, Botulinum Toxin Therapy for Fibromyalgia, U.S. Patent Publication No. 2004/0062776 (Apr. 1, 2004); and Kei Roger Aoki et al., Botulinum Toxin Therapy for Lower Back Pain, U.S. Patent Publication No. 2004/0037852 (Feb. 26, 2004); muscle injuries, see e.g., Gregory F. Brooks, Methods for Treating Muscle Injuries, U.S. Pat. No. 6,423,319 (Jul. 23, 2002); headache, see e.g., Martin Voet, Methods for Treating Sinus Headache, U.S. Pat. No. 6,838,434 (Jan. 4, 2005); Kei Roger Aoki et al., Methods for Treating Tension Headache, U.S. Pat. No. 6,776,992 (Aug. 17, 2004); and Kei Roger Aoki et al., Method for Treating Headache, U.S. Pat. No. 6,458,365 (Oct. 1, 2002); William J. Binder, Method for Reduction of Migraine Headache Pain, U.S. Pat. No. 5,714,469 (Feb. 3, 1998); cardiovascular diseases, see e.g., Gregory F. Brooks and Stephen Donovan, Methods for Treating Cardiovascular Diseases with Botulinum Toxin, U.S. Pat. No. 6,767,544 (Jul. 27, 2004); neurological disorders, see e.g., Stephen Donovan, Parkinson's Disease Treatment, U.S. Pat. No. 6,620,415 (Sep. 16, 2003); and Stephen Donovan, Method for Treating Parkinson's Disease with a Botulinum Toxin, U.S. Pat. No. 6,306,403 (Oct. 23, 2001); neuropsychiatric disorders, see e.g., Stephen Donovan, Botulinum Toxin Therapy for Neuropsychiatric Disorders, U.S. Patent Publication No. 2004/0180061 (Sep. 16, 2004); and Steven Donovan, Therapeutic Treatments for Neuropsychiatric Disorders, U.S. Patent Publication No. 2003/0211121 (Nov. 13, 2003); endocrine disorders, see e.g., Stephen Donovan, Method for Treating Endocrine Disorders, U.S. Pat. No. 6,827,931 (Dec. 7, 2004); Stephen Donovan, Method for Treating Thyroid Disorders with a Botulinum Toxin, U.S. Pat. No. 6,740,321 (May 25, 2004); Kei Roger Aoki et al., Method for Treating a Cholinergic Influenced Sweat Gland, U.S. Pat. No. 6,683,049 (Jan. 27, 2004); Stephen Donovan, Neurotoxin Therapy for Diabetes, U.S. Pat. No. 6,416,765 (Jul. 9, 2002); Stephen Donovan, Methods for Treating Diabetes, U.S. Pat. No. 6,337,075 (Jan. 8, 2002); Stephen Donovan, Method for Treating a Pancreatic Disorder with a Neurotoxin, U.S. Pat. No. 6,261,572 (Jul. 17, 2001); Stephen Donovan, Methods for Treating Pancreatic Disorders, U.S. Pat. No. 6,143,306 (Nov. 7, 2000); cancers, see e.g., Stephen Donovan, Methods for Treating Bone Tumors, U.S. Pat. No. 6,565,870 (May 20, 2003); Stephen Donovan, Method for Treating Cancer with a Neurotoxin to Improve Patient Function, U.S. Pat. No. 6,368,605 (Apr. 9, 2002); Stephen Donovan, Method for Treating Cancer with a Neurotoxin, U.S. Pat. No. 6,139,845 (Oct. 31, 2000); and Mitchell F. Brin and Stephen Donovan, Methods for Treating Diverse Cancers, U.S. Patent Publication No. 2005/0031648 (Feb. 10, 2005); otic disorders, see e.g., Stephen Donovan, Neurotoxin Therapy for Inner Ear Disorders, U.S. Pat. No. 6,358,926 (Mar. 19, 2002); and Stephen Donovan, Method for Treating Otic Disorders, U.S. Pat. No. 6,265,379 (Jul. 24, 2001); autonomic disorders, see, e.g., Pankai J. Pasricha and Anthony N. Kaloo, Method for Treating Gastrointestinal Muscle Disorders and Other Smooth Muscle Dysfunction, U.S. Pat. No. 5,437,291 (Aug. 1, 1995); as well as other disorders, see e.g., William J. Binder, Method for Treatment of Skin Lesions Associated with Cutaneous Cell-proliferative Disorders, U.S. Pat. No. 5,670,484 (Sep. 23, 1997); Eric R. First, Application of Botulinum Toxin to the Management of Neurogenic Inflammatory Disorders, U.S. Pat. No. 6,063,768 (May 16, 2000); Marvin Schwartz and Brian J. Freund, Method to Reduce Hair Loss and Stimulate Hair Growth, U.S. Pat. No. 6,299,893 (Oct. 9, 2001); Jean D. A. Carruthers and Alastair Carruthers, Cosmetic Use of Botulinum Toxin for Treatment of Downturned Mouth, U.S. Pat. No. 6,358,917 (Mar. 19, 2002); Stephen Donovan, Use of a Clostridial Toxin to Reduce Appetite, U.S. Patent Publication No. 2004/40253274 (Dec. 16, 2004); and Howard I. Katz and Andrew M. Blumenfeld, Botulinum Toxin Dental Therapies and Procedures, U.S. Patent Publication No. 2004/0115139 (Jun. 17, 2004); Kei Roger Aoki, et al., Treatment of Neuromuscular Disorders and Conditions with Different Botulinum, U.S. Patent Publication No. 2002/0010138 (Jan. 24, 2002); and Kei Roger Aoki, et al., Use of Botulinum Toxins for Treating Various Disorders and Conditions and Associated Pain, U.S. Patent Publication No. 2004/0013692 (Jan. 22, 2004). In addition, the expected use of Clostridial toxins, such as, e.g., BoNTs, like., BoNT/A, BoNT/B, BoNT/C1, BoNT/D, BoNT/E, BoNT/F and BoNT/G, and TeNT, in therapeutic and cosmetic treatments of humans and other mammals is anticipated to expand to an ever widening range of diseases and aliments that can benefit from the properties of these toxins.

The growing clinical, therapeutic and cosmetic use of Clostridial toxins necessitates the pharmaceutical industry to use accurate assays for Clostridial toxin effects in order to, e.g., ensure accurate pharmaceutical formulations, monitor established quality control standards and evaluate medical treatment regimes. In addition, while Clostridial toxins are being used for a wide range of clinical, therapeutic and cosmetic interventions, current methods for assessing the degree of effect due to toxin administration are often rudimentary and subjective. For example, such methods often rely on observed clinical effects or visual inspection of muscle tone or activity or invasive techniques that measure neuronal activity. The present invention provides novel methods for determining more precisely the administration sites of a Clostridial toxin to a mammal, as well as, methods for assessing the effects of a Clostridial toxin administration in a mammal. These and related advantages are useful for various clinical, therapeutic and cosmetic applications, such as, e.g. the treatment of neuromuscular disorders, neuropathic disorders, eye disorders, pain, muscle injuries, headache, cardiovascular diseases, neuropsychiatric disorders, endocrine disorders, cancers, otic disorders, hyperkinetic facial lines, as well as, other disorders where a Clostridial toxin administration to a mammal can produce a beneficial effect.

### BRIEF DESCRIPTION OF THE DRAWINGS

FIG. 1 shows the schematic representation of injection sites for BoNT/A and capsaicin, and orientation of the laser blood flow monitor on the rat paw.

FIG. 2 shows pretreatment of the rat paw with BoNT/A dose-dependently inhibits capsaicin-induced vasodilatation. FIG. 2A shows mean percentile changes of blood flow in saline-treated rats induced by capsaicin injection (1%, 15 uL volume), in anesthetized rats. FIG. 2B shows averages of area below the curves of percent changes of mean blood flow. Subcutaneous pre-treatment with BoNT/A in the hindpaw plantar surface at doses of 1.0, 3.5, 7.0 and 15.0 U/kg significantly inhibited the capsaicin-induced increase in blood flow, (assessed by one-way ANOVA and post-hoc t-test). While partial inhibition was observed at a dose of 0.35 U/kg, it was not found to be statistically significant. FIG. 2C shows mean percentile change of blood flow induced by CGRP injection (50 pmol, 15 ul) in rats. FIG. 2D shows averages of areas-below-the-curve of percent changes of mean blood flow induced by CGRP injection (50 pmol, 15 ul). The exogenous CGRP-mediated increase in blood flow (which represents a vasodilatory action directly on blood vessels rather than CGRP released from the sensory nerve ending) was not reduced by subcutaneous BoNT/A pretreatment of the rat plantar hindpaw at a dose of 15.0 U/kg, in anesthetized rats. T-test analysis did not show statistical difference between saline-treated and BoNT/A-treated groups.

FIG. 3 shows capsaicin-induced blood flow changes in age-matched vehicle and late stage STZ-treated rats. FIG. 3A illustrates the profile for onset and offset of mechanical allodynia in STZ-treated rats. STZ-treated rats gradually display a reversal of allodynia in the absence of exogenous analgesics (spontaneous reversal or hypoalgesia). The late-stage STZ-treated group represents these spontaneously hypoalgesic subjects. FIG. 3B illustrates the mean percentile change in blood flow evoked by an intraplantar capsaicin injection (1%, 15 uL volume). FIG. 3C summarizes the averages of areas-under-the-curve of percentile change in blood flow versus time. The capsaicin-evoked increase in blood flow was significantly greater in vehicle-treated rats than in the late-stage STZ-treated group. Assessment of peripheral blood flow thus suggests dysfunction in peripheral autonomic regulation which, in turn, supports a more generalized peripheral nerve dysfunction, including nociceptive sensory nerves. Although central changes cannot be ruled out, this suggests that nociceptive sensory nerves may be involved in both establishment and maintenance of the chronic pain state. Loss of nociceptive sensory nerve function would also involve loss of neurogenic inflammation. The latter can, therefore, be assessed by measuring evoked (and spontaneous) blood flow.

FIG. 4 shows BoNT/A inhibits capsaicin-induced increases in cutaneous blood flow at lower doses than observed for inhibition of mechanical allodynia. Comparison of doses of BoNT/A (s.c.) required for inhibition of capsaicin-induced increased peripheral (cutaneous) blood flow versus inhibition of capsaicin-induced mechanical allodynia. Maximal inhibition of cutaneous blood flow is observed at doses that are not (or are minimally) analgesic. This differential in observed dose-response demonstrates the greater sensitivity of the blood flow assessment in detecting actions of Botulinum toxins (such as, e.g., BoNT/A) at peripheral sensory nerve endings (such as nociceptive nerve endings).

### DETAILED DESCRIPTION

The present invention provides, in part, novel methods for assessing the physiological activity of a target site being evaluated for potential administration of a Clostridial toxin using immediate changes in blood flow or flux as an index of assessment. These novel methods take advantage of the fact that abnormal physiological activity underling regions that could benefit from an administration of a Clostridial toxin will exhibit blood flow that is different from the blood flow of an area not requiring such a Clostridial toxin treatment. In addition, the present invention provides, in part, novel methods for assessing the effect of an in vivo administration of a Clostridial toxin in a mammal using immediate changes in blood flow or flux as an index of toxin activity. These novel methods rely on the difference in blood flow from an area affected by a Clostridial toxin as compared to the blood flow from an area unaffected by the toxin. Such differences can be useful, e.g., in assessing which particular area or areas in a mammal should be administered a Clostridial toxin; in administering a Clostridial toxin to a particular area or areas; in assessing the extent of Clostridial toxin administration and whether additional toxin should be administered; and in assessing the extent of dispersal of a Clostridial toxin from a target area to a non-target area in a mammal.

The present invention further provides, in part, novel methods for assessing the physiological activity of a target site being evaluated for potential administration of a Clostridial toxin using heat dissipation as an index of assessment. These novel methods take advantage of the fact that abnormal physiological activity underling regions that could benefit from an administration of a Clostridial toxin will emit thermal energy that is different from the thermal energy emitted by an area not requiring such a Clostridial toxin treatment. In addition, the present invention provides, in part, novel methods for assessing the effect of an in vivo administration of a Clostridial toxin in a mammal using heat dissipation as an index of toxin activity. These novel methods rely on the difference in thermal energy emitted from an area affected by a Clostridial toxin as compared to the thermal energy emitted from an area unaffected by the toxin. Such differences can be useful, e.g., in assessing which particular area or areas in a mammal should be administered a Clostridial toxin; in administering a Clostridial toxin to a particular area or areas; in assessing the extent of Clostridial toxin administration and whether additional toxin should be administered; and in assessing the extent of dispersal of a Clostridial toxin from a target area to a non-target area in a mammal.

The present invention further provides, in part, novel methods for identifying a neurogenic inflammation inhibitor by evaluating the effects of a potential inhibitor on a target site using either blood flow or heat dissipation as an index of evaluation.

Thus, aspects of the present invention provide methods of assessing a physiological activity of a target site for administration of a Clostridial toxin to a mammal, the method comprising the step of recording blood flow from a surface of the target site in the mammal prior to a Clostridial toxin administration. It is envisioned that a Clostridial toxin can be a BoNT/A, a BoNT/B, a BoNT/C1, a BoNT/D, a BoNT/E, a BoNT/F, a BoNT/G, a TeNT, a BaNT or a BuNT.

Other aspects provide methods of administering a Clostridial toxin to a target site in a mammal, the method comprising the steps of recording blood flow from a surface of the target site in the mammal before administration of a Clostridial toxin; and administering the Clostridial toxin to the target site. As a non-limiting example, examining blood flow will identify a target site, thereby provide information regarding where to administer a Clostridial toxin in a mammal.

Other aspects provide methods of assessing the effect of a Clostridial toxin to a target site in a mammal, the method comprising the steps of a) recording a first blood flow from a surface of the target site in the mammal before administration of the Clostridial toxin; b) recording a second blood flow from the surface of the target site in the mammal after administration of the Clostridial toxin; and c) comparing the first blood flow of step (a) to the second blood flow of step (b). As a non-limiting example, a particular toxin parameter, such as, e.g., the efficacy of the toxin, the stability of a toxin or the effectiveness of the toxin, can be determined by assessing the effect of a Clostridial toxin administration in a mammal. As another non-limiting example, a particular treatment parameter, such as, e.g., safety margins of a treatment, degree of success of the treatment or the identification of the location of a subsequent toxin administration, can be determined by assessing the effect of a Clostridial toxin administration in a mammal. As another non-limiting example, a particular intra-target parameter, such as, e.g., the distribution of toxin effect within one muscle, skin region, organ or gland, can be determined by assessing the effect of a Clostridial toxin administration in a mammal. As yet another non-limiting example, the effective lethal dose of a Clostridial toxin formulation can be determined by assessing the effect of a Clostridial toxin administration in a mammal. As yet another non-limiting example, immunoresistance to a Clostridial toxin can be determined by assessing the effect of a Clostridial toxin administration in a mammal.

Other aspects provide methods of assessing dispersal of a Clostridial toxin from a target site to a non-target site in a mammal, the method comprising the steps of a) recording a first blood flow from a surface of the target site in the mammal and a first blood flow from a surface of the non-target site in the mammal before administration of the Clostridial toxin; b) recording a second blood flow from the surface of the target site in the mammal and a second blood flow from the surface of the non-target site in the mammal after administration of the Clostridial toxin; and c) comparing the first blood flow of the target site and the first blood flow of the non-target site of step (a) to the second blood flow of the target site and the second blood flow of the non-target site of step (b). As a non-limiting example, local diffusion of a Clostridial toxin in a mammal can be determined by assessing the dispersal of a Clostridial toxin from a target site to a non-target site by comparing blood flow differences between the two sites. As another non-limiting example, systemic diffusion of a Clostridial toxin in a mammal can be determined by assessing the dispersal of a Clostridial toxin from a target site to a non-target site by comparing blood flow differences between the two sites.

Other aspects provide methods of identifying a neurogenic inflammation inhibitor, the method comprising the steps of a) administering to a mammal an effective amount of a neurogenic inflammation inhibitor to a target site and a control treatment to a non-target site; b) administering an effective amount of a challenger to the target site and to the non-target site, wherein the challenger is administered after the administration of the neurogenic inflammation inhibitor; and c) recording the blood flow in the target site and non-target site, wherein a lower blood flow in the target site as compared to the non-target site is indicative of a neurogenic inflammation inhibitor. In an aspects of this method, the present invention discloses, in part, methods of assessing an inhibitory effect of a Clostridial toxin on neurogenic inflammation in a target site of a mammal, the method comprising the steps of a) administering to a mammal an effective amount of a Clostridial toxin to a target site and a control treatment to a non-target site; b) administering an effective amount of a challenger to the target site and to the non-target site, wherein the challenger is administered after the administration of the Clostridial toxin; and c) recording the blood flow in the target site and non-target site, wherein a lower blood flow in the target site as compared to the non-target site is indicative of a Clostridial toxin effect on neurogenic inflammation. It is envisioned that a challenger can be an Aα-fiber agonist, an Aβ-fiber agonist, an Aγ-fiber agonist, an Aδ-fiber agonist, or a C-fiber agonist. In other aspects of this method, the present invention discloses, in part, methods of assessing an inhibitory effect of a Clostridial toxin on pain in a target site of a mammal, the method comprising the steps of a) administering to a mammal an effective amount of a Clostridial toxin to a target site and a control treatment to a non-target site; b) administering an effective amount of a challenger to the target site and to the non-target site, wherein the challenger is administered after the administration of the Clostridial toxin; and c) recording the blood flow in the target site and non-target site, wherein a lower blood flow in the target site as compared to the non-target site is indicative of a Clostridial toxin effect on pain. It is envisioned that a challenger can be an Aα-fiber agonist, an Aβ-fiber agonist, an Aγ-fiber agonist, an Aδ-fiber agonist, or a C-fiber agonist.

Yet other aspects of the present invention provide methods of assessing a physiological activity of a target site for administration of a Clostridial toxin to a mammal, the method comprising the step of recording a thermal image from a surface of the target site in the mammal prior to a Clostridial toxin administration.

Yet other aspects provide methods of administering a Clostridial toxin to a target site in a mammal, the method comprising the steps of recording a thermal image from a surface of the target site in the mammal before administration of a Clostridial toxin; and administering the Clostridial toxin to the target site. As a non-limiting example, examining a thermal image will identify a target site, thereby provide information regarding where to administer a Clostridial toxin in a mammal.

Yet other aspects provide methods of assessing the effect of a Clostridial toxin to a target site in a mammal, the method comprising the steps of a) recording a first thermal image from a surface of the target site in the mammal before administration of the Clostridial toxin; b) recording a second thermal image from the surface of the target site in the mammal after administration of the Clostridial toxin; and c) comparing the first thermal image of step (a) to the second thermal image of step (b). As a non-limiting example, a particular toxin parameter, such as, e.g., the efficacy of the toxin, the stability of a toxin or the effectiveness of the toxin, can be determined by assessing the effect of a Clostridial toxin administration in a mammal. As another non-limiting example, a particular treatment parameter, such as, e.g., safety margins of a treatment, degree of success of the treatment or the identification of the location of a subsequent toxin administration, can be determined by assessing the effect of a Clostridial toxin administration in a mammal. As another non-limiting example, a particular intra-target parameter, such as, e.g., the distribution of toxin effect within one muscle, skin region, organ or gland, can be determined by assessing the effect of a Clostridial toxin administration in a mammal. As yet another non-limiting example, the effective lethal dose of a Clostridial toxin formulation can be determined by assessing the effect of a Clostridial toxin administration in a mammal. As yet another non-limiting example, immunoresistance to a Clostridial toxin can be determined by assessing the effect of a Clostridial toxin administration in a mammal.

Yet other aspects provide methods of assessing dispersal of a Clostridial toxin from a target site to a non-target site in a mammal, the method comprising the steps of a) recording a first thermal image from a surface of the target site in the mammal and a first thermal image from a surface of the non-target site in the mammal before administration of the Clostridial toxin; b) recording a second thermal image from the surface of the target site in the mammal and a second thermal image from the surface of the non-target site in the mammal after administration of the Clostridial toxin; and c) comparing the first thermal image of the target site and the first thermal image of the non-target site of step (a) to the second thermal image of the target site and the second thermal image of the non-target site of step (b). As a non-limiting example, local diffusion of a Clostridial toxin in a mammal can be determined by assessing the dispersal of a Clostridial toxin from a target site to a non-target site using heat dissipation differences between the two sites. As another non-limiting example, systemic diffusion of a Clostridial toxin in a mammal can be determined by assessing the dispersal of a Clostridial toxin from a target site to a non-target site using heat dissipation differences between the two sites.

Aspects of the present invention provide methods of assessing a physiological activity of a target site for administration of a Clostridial toxin to a mammal. As used herein, the term “mammal” includes, but not limited to, rodents, rabbits, porcines, bovines, equines, non-human primates and humans. As a non-limiting example, a target site for administering a Clostridial toxin can be identified by assessing a physiological activity of a target site in a mammal using blood flow. As a non-limiting example, a target site for administering a Clostridial toxin can be identified by assessing a physiological activity of a target site in a mammal using heat dissipation.

Aspects of the present invention provide, in part, assessing a physiological activity. As used herein, the term “physiological activity” means any process that changes the blood flow in a vessel and/or generates heat resulting in the emission of thermal energy from a surface in a mammal. As used herein, the term “surface” means any body area that can emit thermal energy, such as, e.g., a skin surface or a surface of an exposed internal body part like a muscle, organ or gland. Many physiological activities can generate heat, such as, e.g., a metabolic activity, a neuronal activity, a hemodynamic activity and a muscle activity. Metabolic activities includes, without limitation, an anabolic activity and a catabolic activity. Neuronal activities includes, without limitation, an autonomic neuronal activity; a motor neuronal activity; and a sensory neuronal activity, involving, e.g., a nociceptive stimuli and a non-nociceptive stimuli, like, a chemical stimuli, a thermal stimuli and a mechanical stimuli. As heat is generated by physiological activity in a mammal, it is distributed throughout the body by the circulating blood. Since the interior body temperature of a mammal is usually higher than the surrounding ambient temperature, a temperature gradient produces heat flow from the inside of the body's core to the body's surface. The extent of this temperature gradient is regulated by the blood flow to the surface. As a non-limiting example, vasodilation of the capillaries at the skin surface increases blood flow, which in turn, increases the conduction of heat, thereby increasing the amount of thermal energy emitted from the skin surface. Vasoconstriction of the capillaries in the skin decrease blood flow, which in turn, decrease the conduction of heat, thereby decreasing the amount of thermal energy emitted from the skin surface.

It is envisioned that any disease or disorder benefiting from a Clostridial toxin treatment which exhibits a disrupted physiological activity that results in the emission of thermal energy that is different than a non-disease or non-disorder state can be assessed using methods disclosed in the present specification. Non-limiting examples of such diseases and disorders include, e.g., neuromuscular disorders, neuropathic disorders, movement disorders, eye disorders, pain, muscle injuries, headache, cardiovascular diseases, neuropsychiatric disorders, endocrine disorders, cancers, otic disorders and myokinesis disorders. As a non-limiting example, a muscle undergoing hyperkinesia or muscle spasm, such as, e.g., focal dystonias like blepharospasm, oromandubular dystonia, spasmodic dystonia, cervical dystonia, task-specific dystonias, segmental dystonias, general dystonia, myoclonus, tics and tremors, exhibits physiological activity, such as, e.g., a motor neuronal activity, different than a muscle not experiencing hyperkinesia or muscle spasm. This difference in physiological activity results in a different amount of thermal energy being emitted from the muscle undergoing hyperkinesia or muscle spasm as compared to the muscle not experiencing hyperkinesia or muscle spasm. A thermal image will reveal the muscle undergoing hyperkinesia or muscle spasm, and thus, identify a region that can potentially be treated by administering a Clostridial toxin. As another non-limiting example, abnormal control in axillary sweat gland function resulting in sweating beyond what is physiological necessary to maintain normal thermoregulation, such as, e.g., primary hyperhidrosis, secondary hyperhydrosis and idiopathic hyperhydrosis exhibiting a physiological activity, such as, e.g., an autonomic neuronal activity, different than a normally functioning sweat gland. A thermal image will reveal the sweat gland undergoing abnormal sweating, and thus, identify a region that can potentially be treated by administering a Clostridial toxin. As another non-limiting example, a body region experiencing pain, such as, e.g., inflammatory pain and neuropathic pain, exhibits physiological activity, such as, e.g., a sensory neuronal activity, different than a body region not experiencing pain. This difference in physiological activity results in a different amount of thermal energy being emitted from the region experiencing pain as compared to the region not experiencing pain. A thermal image will reveal the body region experiencing pain, and thus, identify a region that can potentially be treated by administering a Clostridial toxin.

Thus, in an embodiment, a target site is assessed for a physiological activity by recording blood flow in a mammal. In another embodiment, a target area is assessed for metabolic activity by recording blood flow in a mammal. In aspects of this embodiment, a target area is assessed for, e.g., anabolic activity by recording blood flow in a mammal or catabolic activity by recording blood flow in a mammal. In another embodiment, a target area is assessed for neuronal activity by recording blood flow in a mammal. In aspects of this embodiment, a target area is assessed for, e.g., autonomic neuronal activity by recording blood flow in a mammal, motor neuronal activity by recording blood flow in a mammal or sensory neuronal activity by recording blood flow in a mammal. In further aspects of this embodiment, a target area is assessed for, e.g., sensory neuronal activity involving a nociceptin stimulus by recording blood flow in a mammal or sensory neuronal activity involving a non-nociceptin stimulus by recording blood flow in a mammal. In other aspects of this embodiment, a target area is assessed for, e.g., a sensory neuronal activity involving a chemical stimulus by recording blood flow in a mammal, a sensory neuronal activity involving a thermal stimulus by recording blood flow or a sensory neuronal activity involving a mechanical stimulus by recording blood flow in a mammal. In yet another embodiment, a target area is assessed for hemodynamic activity by recording blood flow in a mammal. In a further embodiment, a target area is assessed for muscle activity by recording blood flow in a mammal. In a further embodiment, a target area is assessed for organ activity by recording blood flow in a mammal. In a further embodiment, a target area is assessed for glandular activity by recording blood flow in a mammal.

In another embodiment, a target site is assessed for a physiological activity by recording a thermal image of a surface in a mammal. In another embodiment, a target area is assessed for metabolic activity by recording a thermal image of a surface in a mammal. In aspects of this embodiment, a target area is assessed for, e.g., anabolic activity by recording a thermal image of a surface or catabolic activity by recording a thermal image of a surface. In another embodiment, a target area is assessed for neuronal activity by recording a thermal image of a surface in a mammal. In aspects of this embodiment, a target area is assessed for, e.g., autonomic neuronal activity by recording a thermal image of a surface, motor neuronal activity by recording a thermal image of a surface or sensory neuronal activity by recording a thermal image of a surface. In further aspects of this embodiment, a target area is assessed for, e.g., sensory neuronal activity involving a nociceptin stimulus by recording a thermal image of a surface or sensory neuronal activity involving a non-nociceptin stimulus by recording a thermal image of a surface. In other aspects of this embodiment, a target area is assessed for, e.g., a sensory neuronal activity involving a chemical stimulus by recording a thermal image of a surface, a sensory neuronal activity involving a thermal stimulus by recording a thermal image of a surface or a sensory neuronal activity involving a mechanical stimulus by recording a thermal image of a surface. In yet another embodiment, a target area is assessed for hemodynamic activity by recording a thermal image of a surface in a mammal. In a further embodiment, a target area is assessed for muscle activity by recording a thermal image of a surface in a mammal.

In another embodiment, a target site is assessed for a physiological activity by recording a thermal image of a surface. In an aspect of this embodiment, a target site is assessed for a physiological activity by recording a thermal image of a skin surface. In another aspect of this embodiment, a target site is assessed for a physiological activity by recording a thermal image of a muscle surface. In yet another aspect of this embodiment, a target site is assessed for a physiological activity by recording a thermal image of an organ surface. In still another aspect of this embodiment, a target site is assessed for a physiological activity by recording a thermal image of a gland surface.

Aspects of the present invention provide, in part, assessing a target site. As used herein, the term “target site” means a particular area of a mammalian body for which administration of a Clostridial toxin as a treatment is being considered or is desired. As such, a Clostridial toxin treatment is administered to a target site. Non-limiting examples of a target site can include muscle, such as, e.g., skeletal or striated muscle, smooth muscle like visceral muscle and vascular muscle and cardiac muscle; skin, such as, e.g., epidermis, dermis and subdermis; and organs, such as, e.g., bladder, brain, stomach, pancreas, colon, uterus, thyroid gland, parathyroid gland, prostate gland and sweat glands.

Thus, in an embodiment a target site is assessed for administration of a Clostridial toxin. In aspects of this embodiment, a target site being assessed for administration of a Clostridial toxin can be, e.g., a muscle, a skin region, an organ or a gland. In further aspects of this embodiment, a target muscle site being assessed for administration of a Clostridial toxin can be, e.g., a skeletal muscle, a smooth muscle or a cardiac muscle. In yet further aspects of this embodiment, target skin site being assessed for administration of a Clostridial toxin can be, e.g., epidermal skin, dermal skin, subdermal skin and cutaneous skin or subcutaneous skin. In still further aspects of this embodiment, a target organ site being assessed for administration of a Clostridial toxin can be, e.g., bladder, brain, stomach, pancreas, colon, uterus, thyroid gland, parathyroid gland, prostate gland or sweat gland.

Aspects of the present invention provide, in part, recording blood flow. As used herein, the term “recording blood flow” means detecting blood flow of vessels underlying a target site and/or a non-target site. It is envisioned that any and all techniques that can detect blood flow (flux) can be used, such as, e.g., laser-Doppler techniques, pulsed laser Doppler techniques, transcranial Doppler techniques, plethysmography techniques, and capillaroscopy. Laser-Doppler techniques are used to probe typically the microcirculation of blood in skin tissue with a measurement depth of few millimeters and include, e.g. contact laser-Doppler, scanning laser-Doppler, laser-Doppler fluximetry and laser-Doppler imaging, see, e.g., P. H. Carpentier, New techniques for clinical assessment of the peripheral microcirculation, 58(Spec. No. 1) Drugs 17-22 (1999); A. Lima and J. Bakker, Noninvasive monitoring of peripheral perfusion, 31 (10) Intensive Care Med. 1316-1326 (2005); and G. B. Yvonne-Tee et al., Noninvasive assessment of cutaneous vascular function in vivo using capillaroscopy, plethysmography and laser-Doppler instruments: Its strengths and weaknesses, 34(4) Clin. Hemorheol. Microcirc. 457-473 (2006). Both pulsed laser Doppler techniques and transcranial Doppler techniques allow for measurements at depths of a few centimeters, thereby allowing deeper probing, such as, e.g., brain tissue.

A laser-Doppler technique is a non-invasive, continuous, real-time measurement of microvascular red blood cell (or erythrocyte) perfusion in tissue. Perfusion is sometimes also referred to as microvascular blood flow or red blood cell flux, see, e.g., Bonner, R. F., Clem, T. R., Bowen, P. D., Bowman, R. L., Laser-Doppler Continuous Real-Time Monitor of Pulsatile and Mean Blood Flow in Tissue Microcirculation, in SCATTERING TECHNIQUES, APPLIED TO SUPRA-MOLECULAR AND NONEQUILIBRIUM SYSTEMS (Sow-Hsin Chen, Benjamin Chu, Ralph Nossal, eds., New York, Plenum, 1981). The principle of a Laser-Doppler technique is to measure the frequency change that light undergoes when reflected by moving objects, such as, e.g., red blood cells, called the Doppler shift. A laser-Doppler technique works by illuminating the tissue under observation with low power laser light from a probe containing optical fiber light guides. Laser light from one fiber is scattered within the tissue and some is scattered back to the probe. Another optical fiber collects the backscattered light from the tissue and returns it to the analyzer-recorder. Most of the light is scattered by tissue that is not moving but a small percentage of the returned light is scattered by moving red blood cells. The light returned to the monitor undergoes signal processing whereby the emitted and returned signals are compared to extract the Doppler shift related to moving red blood cells. This output value constitutes the flux of red cells, defined as the number of red cells times their velocity and is reported as microcirculatory perfusion units. A laser-Doppler technique typically uses monochromatic light emitted from a low power laser and the beam can penetrate unbroken, non-pigmented tissue to a depth of 1-2 mm, although depths of serveral centimeters can be acheived using certain laser-Doppler techniques. Measurement of red blood cell motion can be recorded continuously in the outer layer of the tissue under study, with little or no influence on physiologic blood flow. No direct information concerning oxygen, nutrient or waste metabolite exchange in the surrounding tissue is obtained with this technique. The relationship between the flowmeter output signal and the flux of red blood cells is linear.

Aspects of the present invention provide, in part, recording a thermal image of a surface. As used herein, the term “recording a thermal image” means detecting the thermal energy emitted from a target site and/or a non-target site. It is envisioned that any and all thermographic systems that can record a thermal image can be used, such as, e.g., liquid crystal thermography (LCT), infrared thermography (IRT), microwave thermography (MWT) and Computerized thermal imaging (CTI). In general, thermographic systems, use an infrared sensor to convert thermal energy into electric signals thereby producing a thermal image. The thermal image can be generated by means of either an optical scanning system or a pyroelectric vidicon television tube. A video monitor or the like can be used to display the image. Non-limiting examples of thermographic systems include, e.g., Albert F. Kutas and Demetro U. Tokaruk, Scanning Thermography, U.S. Pat. No. 3,862,423 (Jan. 21, 1975); Robert P. Hunt and Richard H. Winkler, Infrared Imaging System, U.S. Pat. No. 3,909,521 (Sep. 30, 1975); Victor J. Anselmo and Terrence H. Reilly, Medical Diagnosis System and Method With Multispectral Imaging, U.S. Pat. No. 4,170,987 (Oct. 16, 1979); Peter T. Walsall and James R. Vincent, Method for Identifying the Presence of Abnormal Tissue, U.S. Pat. No. 4,428,382 (Jan. 31, 1984); Frank K. Leung, Apparatus for Thermographic Examinations, U.S. Pat. No. 4,548,212 ((Oct. 22, 1985); Toshio Murotani, Infrared Imaging Device, U.S. Pat. No. 5,034,794 (Jul. 23, 1991); Akio Tanaka, Infrared Imaging Device and Infrared Imaging System Using Same, U.S. Pat. No. 5,594,248 (Jan. 14, 1997); Zhong Qi Liu and Chen Wang, Method and Apparatus for Thermal Radiation Imaging, U.S. Pat. No. 6,023,637 (Feb. 8, 2000); Liang-Chien Chu and Chih-Chi Chang, Infrared 3D Scanning System, U.S. Pat. No. 6,442,419 (Aug. 27, 2002); and Tae-woo Kim et al., Non-Invasive Apparatus for Measuring A Temperature of A Living Body and Method Therefor, U.S. Pat. No. 6,773,159 (Aug. 10, 2004). In addition, thermographic systems are commercially available, such as, e.g., Teletherm infrared imager (Ashwin Systems International, Inc., Tampa, Fla.); Meditherm med2000™ (Mediterm, Beaufort, N.C.); Thermal Image Processor™ System (Computerized Thermal Imaging, Inc., Ogden, Utah) and TSA ImagIR (Seahorse Bioscience Inc., North Billerica, Mass.).

Thermal imaging, or thermography, visualizes the amount of thermal energy being emitted from a surface. Thermography has been applied in various fields of medicine, veterinary medicine, pharmacy, and dentistry as a valuable diagnostic tool that can potentially differentiate between a diseased and a non-diseased state. These applications take advantage of the fact that surface temperature of the body reflects the activity of underlying physiological processes and their effects on blood circulation. For example, the surface temperature distribution of the skin in a healthy mammalian body exhibits a bilateral symmetry, whereas perturbations in a physiological activity underlying a particular disease or disorder can be associated with an abnormal thermal pattern of the surface, i.e., the loss of bilateral symmetry in the thermal pattern. Thus, a physiological dysfunction can be revealed by either an increase or a decrease in the amount of thermal energy being emitted from the body surface. Current medical applications of thermographic systems include, e.g., detection of blood flow as applied in, e.g., coronary artery bypass surgery, microsurgery, wound healing, peripheral vascular disorders and deep vein thrombosis; staging and analysis of burn trauma; inflammatory diseases; reproductive problems; cancer risk assessment and prognosis; diabetes; pain; neurological problems; neuro-musculoskeletal diseases; and autonomic nervous diseases. Thermal imaging is, therefore, an effective technique for examining both normal and abnormal physiological changes and responses.

The skin temperature varies dynamically and continuously depending on the thermoregulatory state of the mammal. During a resting condition, the body and ambient temperature are allowed to equilibrate to some extent which causes the skin capillaries to vasoconstrict in an effort to conserve thermal energy and maintain the core temperature of the body. During a non-resting condition, a stress is applied to the body which causes the skin capillaries to vasodilate in an effort to release thermal energy and reduce the core temperature of the body. Non-resting conditions can be induced by, without limitation, a thermal stress, such as, e.g., cooling or heating, mechanical stress, such as, e.g., vibration or physical exertion, or chemical stress, such as, e.g., vasodilators or vasoconstrictors. Thus, a resting condition will reflect a certain thermoregulatory state whereas a non-resting condition will reflect a different thermoregulatory state from that of the resting condition. It is understood that a resting condition may not always produce a maximal difference in the thermal energy emitted. Thus, in order to exacerbate the difference in thermal energy being emitted from a region exhibiting an abnormal physiological activity as compared to a region exhibiting normal physiological activity, a thermal image of a mammal may be taken under non-resting conditions.

Thus, in one embodiment, a thermal image recording can be done during a resting condition. In another embodiment, a plurality of thermal image recordings can be done during a resting condition. In yet another embodiment, a thermal image recording can be done during a non-resting condition. In yet another embodiment, a plurality of thermal image recordings can be done during a non-resting condition. As a non-limiting example of a resting condition, the target area is exposed to the environment, e.g., by removing any clothing or shaving away fur, and the mammal takes a comfortable, relaxing position in a climate controlled room held at approximately 18-22±1° C. for a period of approximately 10-30 minutes. As a non-limiting example of a non-resting condition, the target area is exposed to the environment, e.g., by removing any clothing or shaving away fur, and the mammal undergoes physical exertion, such as, e.g., running in place, on a read mill, on an exercise wheel, in a climate controlled room held at approximately 18-22±1° C. for a period of approximately 5-30 minutes.

Thermal imaging can record thermal energy over the entire body surface of a mammal to detect systemic thermal variation or this technique can record thermal energy of a discrete body surface to detect localized thermal variation. Thus, in one embodiment, recording of a thermal image can be done over an entire body surface of a mammal to detect systemic thermal variation. In another embodiment, recording of a thermal image can be done at a discrete body surface to detect localized thermal variation.

Aspects of the present invention comprise, in part, a Clostridial toxin. As used herein, the term “Clostridial toxin” means a naturally-occurring Clostridial toxin or non-naturally occurring Clostridial toxin. Naturally-occurring Clostridial toxins are found in many species belonging to the genus Clostridium, including, without limitation, C. botulinum, C. tetani, C. baratii and C. butyricum. Seven antigenically-distinct serotypes of Botulinum toxins (BoNTs) have been identified by investigating botulism outbreaks in man (BoNT/A, /B, /E and /F), animals (BoNT/C1 and /D), or isolated from soil (BoNT/G). It is recognized by those of skill in the art that within each type of Clostridial toxin there can be subtypes that differ somewhat in their amino acid sequence, and also in the nucleic acids encoding these proteins. For example, BoNT/A subtypes include, e.g., BoNT/A1, BoNT/A2, BoNT/A3 and BoNT/A4; BoNT/B subtypes include, e.g., BoNT/B1, BoNT/B2, BoNT/B bivalent and BoNT/B nonproteolytic; BoNT/C1 subtypes include, e.g., BoNT/C1-1 and BoNT/C1-2; and BoNT/E subtypes include BoNT/E1, BoNT/E2 and BoNT/E3. Tetanus toxin (TeNT) appears to be produced by a uniform group of C. tetani, while C. baratii (BaNT) and C. butyricum (BuNT), also produce toxins similar to BoNT/F and BoNT/E, respectively. Clostridial toxins commercially available as pharmaceutical compositions include, BoNT/A preparations, such as, e.g., BOTOX® (Allergan, Inc., Irvine, Calif.), Dysport®/Reloxin®, (Beaufour Ipsen, Porton Down, England), Linurasee (Prollenium, Inc., Ontario, Canada), Neuronoxe (Medy-Tox, Inc., Ochang-myeon, South Korea) BTX-A (Lanzhou Institute Biological Products, China) and Xeomine (Merz Pharmaceuticals, GmbH., Frankfurt, Germany); and BoNT/B preparations, such as, e.g., MyoBloc®/NeuroBloce (Elan Pharmaceuticals, San Francisco, Calif.).

Modified Clostridial toxins include active fragments, chimeras, and other recombinant derivatives useful for clinical, therapeutic and cosmetic applications. Such toxins are disclosed in, e.g., Lance E. Steward et al., Modified Clostridial Toxins with Enhanced Targeting Capabilities For Endogenous Clostridial Toxin Receptor Systems, International Patent Application No. 2006/008956; Lance E. Steward et al., Modified Clostridial Toxins with Altered Targeting Capabilities For Clostridial Toxin Target Cells, International Patent Application No. 2006/009831 (Mar. 15, 2005); Lance E. Steward et al., Multivalent Clostridial Toxin Derivatives and Methods of Their Use, U.S. Patent Publication 2006/0211619 (Sep. 21, 2006); Keith A. Foster et al., Clostridial Toxin Derivatives Able To Modify Peripheral Sensory Afferent Functions, U.S. Pat. No. 5,989,545 (Nov. 23, 1999); Clifford C. Shone et al., Recombinant Toxin Fragments, U.S. Pat. No. 6,461,617 (Oct. 8, 2002); Conrad P. Quinn et al., Methods and Compounds for the Treatment of Mucus Hypersecretion, U.S. Pat. No. 6,632,440 (Oct. 14, 2003); Lance E. Steward et al., Methods And Compositions For The Treatment Of Pancreatitis, U.S. Pat. No. 6,843,998 (Jan. 18, 2005); J. Oliver Dolly et al., Activatable Recombinant Neurotoxins, U.S. Pat. No. 7,132,259 (Nov. 7, 2006); Stephan Donovan, Clostridial Toxin Derivatives and Methods For Treating Pain, U.S. Patent Publication 2002/0037833 (Mar. 28, 2002); Stephan Donovan, Clostridial Toxin Derivatives and Methods For Treating Pain, U.S. Patent Publication US 2006/0093625 (May 4, 2006); Keith A. Foster et al., Inhibition of Secretion from Non-Neural Cells, U.S. Patent Publication 2003/0180289 (Sep. 25, 2003); and Keith A. Foster et al., Re-targeted Toxin Conjugates, International Patent Publication WO 2005/023309 (Mar. 17, 2005), each of which is hereby incorporated by reference in its entirety.

Aspects of the present invention comprise, in part, a Clostridial toxin treatment. As used herein, the term “Clostridial toxin treatment” means a remedy, cure, healing, rehabilitation or any other means of counteracting something undesirable in an individual requiring neuromodulation using a Clostridial toxin or administering to a mammal one or more controlled doses of a medication, preparation or mixture of a Clostridial toxin that has medicinal, therapeutic, curative, cosmetic, remedial or any other beneficial effect. Non-limiting examples of a Clostridial toxin treatment include, e.g., a BoNT/A treatment, a BoNT/B treatment, a BoNT/C1 treatment, a BoNT/D treatment, a BoNT/E treatment, a BoNT/F treatment, a BoNT/G treatment and a TeNT treatment. The term Clostridial toxin treatment encompasses, without limitation, the use of any naturally occurring Clostridial toxin or modified Clostridial toxin, in any formulation, combined with any carrier or active ingredient and administered by any route of administration. Well-known botulinum toxin treatments include, without limitation, a BoNT/A treatment, such as, e.g., a BOTOX® treatment, a Dysport®/Reloxin® treatment, a Linurase® treatment, a Neuronox® treatment, a BTX-A treatment, and a Xeomin® treatment; and a BoNT/B treatment, such as, e.g., a MyoBloc®/NeuroBloc® treatment. Appropriate therapeutic and cosmetic uses of a Clostridial toxin treatment are known in the art. As used herein, the term “individual,” when used in reference to Clostridial toxin treatment, means any organism capable of benefiting from the effects of a Clostridial toxin treatment, including, but not limited to, birds and mammals, including mice, rats, goats, sheep, horses, donkeys, cows, primates and humans.

Aspects of the present invention provide, in part, administration of a Clostridial toxin or Clostridial toxin treatment. As used herein, the term “administration” means any means that provides a Clostridial toxin to a target tissue that potentially results in a clinically, therapeutically, cosmetically or experimentally beneficial result. Administration can be local or systemic. Local administration results in significantly more Clostridial toxin being delivered to a specific location as compared to the entire body of the subject, whereas, systemic administration results in delivery of a Clostridial toxin to essentially the entire body of the subject. Administration of a Clostridial toxin can be by any means including, without limitation, orally in any acceptable form, such as, e.g., tablet, liquid, capsule, powder, or the like; topically in any acceptable form, such as, e.g., patch, drops, creams, gels or ointments; by injection, in any acceptable form, such as, e.g., intravenous, intraperitoneal, intramuscular, subcutaneous, parenteral or epidural; and by implant, such as, e.g., subcutaneous pump, intrathecal pump or other bioerodible or non-bioerodible implanted extended release device or formulation. In general administration of a Clostridial toxin to a mammal can depend on, e.g., the type and location of the disorder, the toxin or other molecule to be included in the composition, and the history, risk factors and symptoms of the mammal.

Thus, in one embodiment, a Clostridial toxin is administered to a target site. In aspects of this embodiment, a Clostridial toxin is administered orally to a target site, a Clostridial toxin is administered topically to a target site, a Clostridial toxin is injected to a target site or a Clostridial toxin is implanted in a target site.

Thus, in an embodiment, a Clostridial toxin is administered to a target site. In aspects of this embodiment, a Botulinum toxin is administered to a target site, a Tetanus toxin is administered to a target site, a C. baratii toxin is administered to a target site or a C. butyricum toxin is administered to a target site. In other aspects of this embodiment, a Clostridial toxin administered to a target site can be, e.g., a BoNT/A, a BoNT/B, a BoNT/C1, a BoNT/D, a BoNT/E, a BoNT/F or a BoNT/G. In still other aspects of this embodiment, a Clostridial toxin administered to a target site can be, e.g., a BOTOX® preparation, a Dysport®/Reloxin® preparation, a Linurase® preparation, a Neuronox® preparation, a BTX-A preparation, a Xeomin® preparation or a MyoBloc™/NeuroBloc™ preparation. In yet other aspects of this embodiment, a Clostridial toxin administered to a target site can be, e.g., a recombinant Clostridial toxin, an active fragment of a Clostridial toxin, a Clostridial toxin derivative or a chimeric Clostridial toxin.

The specific dosage administered to a mammal depends on several factors, including, without limitation, the size and type of the target site to be treated, the type and severity of the disease or disorder to be treated, the weight and age of the mammal, the responsiveness of the mammal to a treatment and the particular commercial preparation of the Clostridial toxin. For example, 18 U/kg total body weight of a BOTOX® preparation, with a per use maximum dose of 400 units are administered to patients suffering from spasticity. Appropriate administration is readily determined by one of ordinary skill in the art according to the factors discussed above. As a non-limiting example, approximately 75-125 units of BOTOX® per intramuscular injection (multiple muscles) are administered to a patient undergoing treatment for cervical dystonia. As another non-limiting example, approximately 5-10 units of BOTOX® per intramuscular injection (5 units injected intramuscularly into the procerus muscle and 10 units injected intramuscularly into each corrugator supercilii muscle) is administered to a patient undergoing treatment for glabellar lines (brow furrows). As another non-limiting example, approximately 30-80 units of BOTOX® is administered to a patient undergoing treatment for constipation by intrasphincter injection of the puborectalis muscle. As yet another non-limiting example, approximately 1-5 units per muscle of intramuscularly injected BOTOX® is administered to a patient undergoing treatment for blepharospasm by injecting the lateral pre-tarsal orbicularis oculi muscle of the upper lid and the lateral pre-tarsal orbicularis oculi of the lower lid. As yet another non-limiting example, approximately 1-5 units of BOTOX® is administered to a patient undergoing treatment for strabismus, the dose of toxin intramuscular injected of the extraocular muscles depending upon both the size of the muscle to be injected and the extent of muscle paralysis desired (i.e. amount of diopter correction desired). As still another non-limiting example, upper limb spasticity following stroke is treated by intramuscular injections of BOTOX® into five different upper limb flexor muscles, as follows: (a) flexor digitorum profundus: 7.5 units to 30 units; (b) flexor digitorum sublimus: 7.5 units to 30 units; (c) flexor carpi ulnaris: 10 units to 40 units; (d) flexor carpi radialis: 15 units to 60 units; (e) biceps brachii: 50 units to 200 units. Each of the five indicated muscles has been injected at the same treatment session, so that the patient receives from 90 units to 360 units of upper limb flexor muscle BOTOX® by intramuscular injection at each treatment session. As still another non-limiting example, approximately 25 units of BOTOX® is administered by pericranial injected (injected symmetrically into glabellar, frontalis and temporalis muscles) to a patient undergoing treatment for migraine.

Aspects of the present invention comprise, in part, administering an effective amount of a Clostridial toxin treatment. As used herein, the term “effective amount” when used in reference to administering a Clostridial toxin treatment means the minimum dose necessary to achieve the desired therapeutic effect and includes a dose sufficient to reduce a symptom associated with a capsaicin response. In aspects of this embodiment, an effect amount of a Clostridial toxin treatment reduces a symptom associated with a capsaicin response by, e.g., at least 30%, at least 40%, at least 50%, at least 60%, at least 70%, at least 80%, at least 90% or at least 100%. In other aspects of this embodiment, an effect amount of a Clostridial toxin treatment reduces a symptom associated with a capsaicin response by, e.g., at most 30%, at most 40%, at most 50%, at most 60%, at most 70%, at most 80%, at most 90% or at most 100%.

The appropriate effective amount to be administered for a particular application of the methods can be determined by those skilled in the art, using the guidance provided herein. For example, an effective amount can be extrapolated from in vitro assays and in vivo administration studies using animal models prior to administration to humans. Such a effect amount generally is in the range of 0.1-1000 mg/day and can be, e.g., in the range of 0.1-500 mg/day, 0.5-500 mg/day, 0.5-100 mg/day, 0.5-50 mg/day, 0.5-20 mg/day, 0.5-10 mg/day or 0.5-5 mg/day. An effective amount of a Clostridial toxin treatment useful for reducing a capsaicin response in an individual will be determined by a one skilled in the pertinent art taking into account the type and amount of Clostridial toxin used, the type and amount of capsaicin used, and the route administration. Where repeated administration is used, the frequency of administration depends, in part, on the half-life of a Clostridial toxin treatment. One skilled in the art will recognize that the condition of the individual can be monitored throughout the course of therapy and that the effective amount of a Clostridial toxin treatment that is administered can be adjusted accordingly. It is also understood that the frequency and duration of dosing will be dependent, in part, on the relief desired and the half-life of a Clostridial toxin treatment.

Aspects of the present invention provide, in part, administering a Clostridial toxin to a target site. Non-limiting examples of a target site that is administered a Clostridial toxin can include muscle, such as, e.g., skeletal or striated muscle, smooth muscle like visceral muscle and vascular muscle and cardiac muscle; skin, such as, e.g., epidermis, dermis and subdermis; and organs, such as, e.g., bladder, brain, stomach, pancreas, colon, uterus, thyroid gland, parathyroid gland, prostate gland and sweat glands.

Thus, in an embodiment a target site is administered a Clostridial toxin. In aspects of this embodiment, a target site that is administered a Clostridial toxin can be, e.g., a muscle, a skin region, an organ or a gland. In further aspects of this embodiment, a target muscle site being administered a Clostridial toxin can be, e.g., a skeletal muscle, a smooth muscle or a cardiac muscle. In yet further aspects of this embodiment, target skin site being administered a Clostridial toxin can be, e.g., epidermal skin, dermal skin, subdermal skin and cutaneous skin or subcutaneous skin. In still further aspects of this embodiment, a target organ or gland site being administered a Clostridial toxin can be, e.g., bladder, brain, stomach, pancreas, colon, uterus, thyroid gland, parathyroid gland, prostate gland or sweat gland.

Aspects of the present invention comprise, in part, a control treatment. As used herein, the term “control treatment” means any treatment lacking the Clostridial toxin treatment being administered to the test site, and includes, e.g., a placebo treatment. In general a control treatment will be identical to a Clostridial toxin treatment except that the control treatment will lack the Clostridial toxin.

Aspects of the present invention comprise, in part, administering a challenger. As used herein, the term “challenger” means any molecule that stimulates a neuron to elicit an inflammatory response, i.e., neurogenic inflammation, and/or a pain response. Non-limiting examples of a challenger include an Aα-fiber agonist; an Aβ-fiber agonist, such as, e.g., diphenyl compounds, lipids, and protons; an Aγ-fiber agonist; an Aδ-fiber agonist, such as, e.g., diphenyl compounds, lipids, protons, endocannabinoids, PUFAs, 4αPDD, epoxyeicosatrienoic acids,; or a C-fiber agonist, such as, e.g., diphenyl compounds, lipids, protons, endocannabinoids, capsaicin, formalin, phenyldiguanide, isothiocyanates, icilin, menthol, camphor, carvacrol, PUFAs, 4αPDD, epoxyeicosatrienoic acids, bradykinin, substance P, and mustard oil, see, e.g., Ellen A. Lumpkin and Michael J. Caterina, Mechanisms of Sensory Transduction in the Skin, 445(7130) Nature 858-865 (2007).

Aspects of the present invention comprise, in part, administering a challenger treatment. As used herein, the term “challenger treatment” means inducing neurogenic inflammation and/or pain by using a challenger.

Aspects of the present invention comprise, in part, administering an effective amount of a challenger treatment. As used herein, the term “effective amount” when used in reference to administering a challenger treatment means the minimum dose necessary to achieve the desired induced sensitization, such as, e.g., neurogenic inflammation and/or pain. In aspects of this embodiment, an effective amount of a challenger treatment induces peripheral sensitization by, e.g., at least 30%, at least 40%, at least 50%, at least 60%, at least 70%, at least 80%, at least 90% or at least 100%. In other aspects of this embodiment, an effective amount of a challenger treatment induces peripheral sensitization by, e.g., at most 30%, at most 40%, at most 50%, at most 60%, at most 70%, at most 80%, at most 90% or at most 100%. In yet other aspects of this embodiment, an effective amount of a challenger treatment induces central sensitization by, e.g., at least 30%, at least 40%, at least 50%, at least 60%, at least 70%, at least 80%, at least 90% or at least 100%. In yet other aspects of this embodiment, an effective amount of a challenger treatment induces central sensitization by, e.g., at most 30%, at most 40%, at most 50%, at most 60%, at most 70%, at most 80%, at most 90% or at most 100%. In still other aspects of this embodiment, an effective amount of a challenger treatment induces both peripheral and central sensitization by, e.g., at least 30%, at least 40%, at least 50%, at least 60%, at least 70%, at least 80%, at least 90% or at least 100%. In still other aspects of this embodiment, an effective amount of a challenger treatment induces both peripheral and central sensitization by, e.g., at most 30%, at most 40%, at most 50%, at most 60%, at most 70%, at most 80%, at most 90% or at most 100%.

In yet other aspects of this embodiment, an effective amount of a challenger treatment is, e.g., at least a 0.01% solution, at least a 0.05% solution, at least a 0.1% solution, at least a 0.5% solution, at least a 1.0% solution. In yet other aspects of this embodiment, an effective amount of a challenger treatment is, e.g., at most a 0.01% solution, at most a 0.05% solution, at most a 0.1% solution, at most a 0.5% solution, at most a 1.0% solution.

Aspects of the present invention comprise, in part, a challenger treatment that is administered after the administration of the Clostridial toxin treatment. It is envisioned that administration of a challenger treatment can occur at any length of time after the administration of the Clostridial toxin treatment, with the proviso that the Clostridial toxin treatment is still in effect. Thus, in aspect of this embodiment, a challenger treatment can occur, e.g., at least one hour after administration of the Clostridial toxin treatment, at least six hours after administration of the Clostridial toxin treatment, at least 12 hours after administration of the Clostridial toxin treatment, at least 24 hour after administration of the Clostridial toxin treatment, at least 48 hour after administration of the Clostridial toxin treatment, at least one week after administration of the Clostridial toxin treatment at least two weeks after administration of the Clostridial toxin treatment or at least one month after administration of the Clostridial toxin treatment. In other aspect of this embodiment, a challenger treatment can occur, e.g., at most one hour after administration of the Clostridial toxin treatment, at most six hours after administration of the Clostridial toxin treatment, at most 12 hours after administration of the Clostridial toxin treatment, at most 24 hour after administration of the Clostridial toxin treatment, at most 48 hour after administration of the Clostridial toxin treatment, at most one week after administration of the Clostridial toxin treatment at most two weeks after administration of the Clostridial toxin treatment or at most one month after administration of the Clostridial toxin treatment.

Aspects of the present invention comprise, in part, a neurogenic inflammation inhibitor. As used herein, the term “neurogenic inflammation inhibitor” means a molecule that that reduces a symptom associated with neurogenic inflammation. In aspects of this embodiment, a neurogenic inflammation inhibitor reduces a symptom associated with neurogenic inflammation by, e.g., at least 10%, at least 20%, at least 30%, at least 40%, at least 50%, at least 60%, at least 70%, at least 80%, at least 90%, or at least 100%. In other aspects of this embodiment, a neurogenic inflammation inhibitor reduces a symptom associated with neurogenic inflammation by, e.g., at least 2-fold, at least 3-fold, at least 4-fold, at least 5-fold, at least 10-fold or at least 20-fold. In yet other aspects of this embodiment, a neurogenic inflammation inhibitor reduces a symptom associated with neurogenic inflammation by, e.g., at most 10%, at most 20%, at most 30%, at most 40%, at most 50%, at most 60%, at most 70%, at most 80%, at most 90%, or at most 100%. In still aspects of this embodiment, a neurogenic inflammation inhibitor reduces a symptom associated with neurogenic inflammation by, e.g., at most 2-fold, at most 3-fold, at most 4-fold, at most 5-fold, at most 10-fold or at most 20-fold. Non limiting examples of a neurogenic inflammation inhibitor include a Clostridial toxin, such as, e.g., a BoNT/A, a BoNT/B, a BoNT/C1, a BoNT/D, a BoNT/E, a BoNT/F, a BoNT/G, a TeNT, a BaNT, and a BuNT. Other non-limiting examples of a neurogenic inflammation inhibitor include modified Clostridial toxins such as those disclosed in, e.g., Lance E. Steward et al., Modified Clostridial Toxins with Enhanced Targeting Capabilities For Endogenous Clostridial Toxin Receptor Systems, International Patent Application No. 2006/008956; Lance E. Steward et al., Modified Clostridial Toxins with Altered Targeting Capabilities For Clostridial Toxin Target Cells, International Patent Application No. 2006/009831 (Mar. 15, 2005); Lance E. Steward et al., Multivalent Clostridial Toxin Derivatives and Methods of Their Use, U.S. Patent Publication 2006/0211619 (Sep. 21, 2006); Keith A. Foster et al., Clostridial Toxin Derivatives Able To Modify Peripheral Sensory Afferent Functions, U.S. Pat. No. 5,989,545 (Nov. 23, 1999); Clifford C. Shone et al., Recombinant Toxin Fragments, U.S. Pat. No. 6,461,617 (Oct. 8, 2002); Conrad P. Quinn et al., Methods and Compounds for the Treatment of Mucus Hypersecretion, U.S. Pat. No. 6,632,440 (Oct. 14, 2003); Lance E. Steward et al., Methods And Compositions For The Treatment Of Pancreatitis, U.S. Pat. No. 6,843,998 (Jan. 18, 2005); J. Oliver Dolly et al., Activatable Recombinant Neurotoxins, U.S. Pat. No. 7,132,259 (Nov. 7, 2006); Stephan Donovan, Clostridial Toxin Derivatives and Methods For Treating Pain, U.S. Patent Publication 2002/0037833 (Mar. 28, 2002); Stephan Donovan, Clostridial Toxin Derivatives and Methods For Treating Pain, U.S. Patent Publication US 2006/0093625 (May 4, 2006); Keith A. Foster et al., Inhibition of Secretion from Non-Neural Cells, U.S. Patent Publication 2003/0180289 (Sep. 25, 2003); and Keith A. Foster et al., Re-targeted Toxin Conjugates, International Patent Publication WO 2005/023309 (Mar. 17, 2005).

Aspects of the present invention comprise, in part, a pain inhibitor. As used herein, the term “pain inhibitor” means a molecule that that reduces a symptom associated with pain. In aspects of this embodiment, a pain inhibitor reduces a symptom associated with pain by, e.g., at least 10%, at least 20%, at least 30%, at least 40%, at least 50%, at least 60%, at least 70%, at least 80%, at least 90%, or at least 100%. In other aspects of this embodiment, a pain inhibitor reduces a symptom associated with pain by, e.g., at least 2-fold, at least 3-fold, at least 4-fold, at least 5-fold, at least 10-fold or at least 20-fold. In yet other aspects of this embodiment, a pain inhibitor reduces a symptom associated with pain by, e.g., at most 10%, at most 20%, at most 30%, at most 40%, at most 50%, at most 60%, at most 70%, at most 80%, at most 90%, or at most 100%. In still aspects of this embodiment, a pain inhibitor reduces a symptom associated with pain by, e.g., at most 2-fold, at most 3-fold, at most 4-fold, at most 5-fold, at most 10-fold or at most 20-fold. Non limiting examples of a pain inhibitor include a Clostridial toxin, such as, e.g.,, a BoNT/A, a BoNT/B, a BoNT/C1, a BoNT/D, a BoNT/E, a BoNT/F, a BoNT/G, a TeNT, a BaNT, and a BuNT. Other non-limiting examples of a pain inhibitor include modified Clostridial toxins such as those disclosed in, e.g., Lance E. Steward et al., Modified Clostridial Toxins with Enhanced Targeting Capabilities For Endogenous Clostridial Toxin Receptor Systems, International Patent Application No. 2006/008956; Lance E. Steward et al., Modified Clostridial Toxins with Altered Targeting Capabilities For Clostridial Toxin Target Cells, International Patent Application No. 2006/009831 (Mar. 15, 2005); Lance E. Steward et al., Multivalent Clostridial Toxin Derivatives and Methods of Their Use, U.S. Patent Publication 2006/0211619 (Sep. 21, 2006); Keith A. Foster et al., Clostridial Toxin Derivatives Able To Modify Peripheral Sensory Afferent Functions, U.S. Pat. No. 5,989,545 (Nov. 23, 1999); Clifford C. Shone et al., Recombinant Toxin Fragments, U.S. Pat. No. 6,461,617 (Oct. 8, 2002); Conrad P. Quinn et al., Methods and Compounds for the Treatment of Mucus Hypersecretion, U.S. Pat. No. 6,632,440 (Oct. 14, 2003); Lance E. Steward et al., Methods And Compositions For The Treatment Of Pancreatitis, U.S. Pat. No. 6,843,998 (Jan. 18, 2005); J. Oliver Dolly et al., Activatable Recombinant Neurotoxins, U.S. Pat. No. 7,132,259 (Nov. 7, 2006); Stephan Donovan, Clostridial Toxin Derivatives and Methods For Treating Pain, U.S. Patent Publication 2002/0037833 (Mar. 28, 2002); Stephan Donovan, Clostridial Toxin Derivatives and Methods For Treating Pain, U.S. Patent Publication US 2006/0093625 (May 4, 2006); Keith A. Foster et al., Inhibition of Secretion from Non-Neural Cells, U.S. Patent Publication 2003/0180289 (Sep. 25, 2003); and Keith A. Foster et al., Re-targeted Toxin Conjugates, International Patent Publication WO 2005/023309 (Mar. 17, 2005).

Aspects of the present invention provide, in part, recording blood flow or a thermal image before administration of a Clostridial toxin. As used herein, the term “before” means any length of time prior to the actual administration of a Clostridial toxin to a mammal. In one embodiment, the recording of blood flow or a thermal image occurs before administration of a Clostridial toxin. Aspects of this embodiment include recording blood flow or a thermal image, e.g., at least one minute before administration of a Clostridial toxin, at least 5 minutes before administration of a Clostridial toxin, at least 15 minutes before administration of a Clostridial toxin, at least 30 minutes before administration of a Clostridial toxin, at least 45 minutes before administration of a Clostridial toxin or at least 60 minutes before administration of a Clostridial toxin. Other aspects of this embodiment include recording blood flow or a thermal image, e.g., at least one hour before administration of a Clostridial toxin, at least two hours before administration of a Clostridial toxin, at least four hours before administration of a Clostridial toxin, at least eight hours before administration of a Clostridial toxin, at least 12 hours before administration of a Clostridial toxin or at least 24 hours before administration of a Clostridial toxin. Further aspects of this embodiment include recording blood flow or a thermal image, e.g., at least one day before administration of a Clostridial toxin, at least two days before administration of a Clostridial toxin, at least four days before administration of a Clostridial toxin, at least eight days before administration of a Clostridial toxin, at least 15 days before administration of a Clostridial toxin or at least 30 days before administration of a Clostridial toxin.

Additional aspects of this embodiment include recording blood flow or a thermal image, e.g., at most one minute before administration of a Clostridial toxin, at most 5 minutes before administration of a Clostridial toxin, at most 15 minutes before administration of a Clostridial toxin, at most 30 minutes before administration of a Clostridial toxin, at most 45 minutes before administration of a Clostridial toxin or at most 60 minutes before administration of a Clostridial toxin. Still other aspects of this embodiment include recording blood flow or a thermal image, e.g., at most one hour before administration of a Clostridial toxin, at most two hours before administration of a Clostridial toxin, at most four hours before administration of a Clostridial toxin, at most eight hours before administration of a Clostridial toxin, at most 12 hours before administration of a Clostridial toxin or at most 24 hours before administration of a Clostridial toxin. Still further aspects of this embodiment include recording blood flow or a thermal image, e.g., at most one day before administration of a Clostridial toxin, at most two days before administration of a Clostridial toxin, at most four days before administration of a Clostridial toxin, at most eight days before administration of a Clostridial toxin, at most 15 days before administration of a Clostridial toxin or at most 30 days before administration of a Clostridial toxin.

Aspects of the present invention provide, in part, assessing the effect of a Clostridial toxin. As used herein, the term “effect” means a change in a physiological activity that is a direct or indirect result of a Clostridial toxin activity, such as, e.g., disruption of a SNARE-mediated process. Non-limiting examples of a Clostridial toxin effect can include, e.g., inhibiting the release of a neuronal molecule, such as, e.g., a neurotransmitter, a neuromodulator, a neuropeptide or a neurohormone; inhibiting the release of a non-neuronal molecule, such as, e.g., a growth factor, a cytokine, a hormone, an enzyme or a lipid; inhibiting an activity of, e.g., a muscle, a skin region, an organ or a gland. In vitro studies indicated that Clostridial toxins inhibit potassium cation induced release of both acetylcholine and norepinephrine from primary cell cultures of brainstem tissue; inhibit the evoked release of both glycine and glutamate in primary cultures of spinal cord neurons; and inhibit the release of acetylcholine, dopamine, norepinephrine, CGRP, substance P and glutamate in brain synaptosome preparations. The neuronal molecules listed above, mediate a wide range of neuronal activities including, without limitation, autonomic neuronal activity; motor neuronal activity; and sensory neuronal activity. As a non-limiting example, a therapeutically effective amount of BoNT/A administered into the underlying facial muscles inhibits the release of the neurotransmitter acetylcholine at the neuromuscular junction thereby relieving hyperkinetic facial lines of the forehead. As another non-limiting example, a therapeutically effective amount of BoNT/A administered into the sweat glands inhibits the release of neurotransmitters from the autonomic neurons controlling sweat release, thereby reducing the symptoms of hyperhidrosis. As yet another non-limiting example, a therapeutically effective amount of BoNT/A administered into the skin reduces the pain response evoked by sensory neurons and local vasomotor reaction of the surrounding blood vessels.

Thus, in an embodiment a target site is assessed for a Clostridial toxin effect. In aspects of this embodiment, a Clostridial toxin effect is assessed by a change in, e.g., a release of a neuronal molecule, a release of a non-neuronal molecule or an activity of a muscle, a skin region, an organ or a gland. In further aspects of this embodiment, a Clostridial toxin effect is assessed by a change in a release of a neuronal molecule, such as, e.g., a neurotransmitter, a neuromodulator, a neuropeptide or a neurohormone. In yet further aspects of this embodiment, a Clostridial toxin effect is assessed by a change in a release of a non-neuronal molecule, such as, e.g., a growth factor, a cytokine, a hormone, an enzyme or a lipid.

Aspects of the present invention provide, in part, assessing the effect of a Clostridial toxin by recording blood flow or a thermal image from a surface of a target site. Non-limiting examples of a surface that blood flow or a thermal image can be recorded can include, e.g., a skin surface, a muscle surface, an organ surface or a gland surface. Non-limiting examples of a target site being assessed for a Clostridial toxin effect can include muscle, such as, e.g., skeletal or striated muscle, smooth muscle like visceral muscle and vascular muscle and cardiac muscle; skin, such as, e.g., epidermis, dermis and subdermis; and organs, such as, e.g., bladder, brain, stomach, pancreas, colon, uterus, thyroid gland, parathyroid gland, prostate gland and sweat glands.

Thus, in an embodiment a target site is assessed for a Clostridial toxin effect. In aspects of this embodiment, a target site being assessed for a Clostridial toxin effect can be, e.g., a muscle, a skin region, an organ or a gland. In further aspects of this embodiment, a target muscle site being assessed for a Clostridial toxin effect can be, e.g., a skeletal muscle, a smooth muscle or a cardiac muscle. In yet further aspects of this embodiment, target skin site being assessed for a Clostridial toxin effect can be, e.g., epidermal skin, dermal skin, subdermal skin and cutaneous skin or subcutaneous skin. In still further aspects of this embodiment, a target organ or gland site being assessed for a Clostridial toxin effect can be, e.g., bladder, brain, stomach, pancreas, colon, uterus, thyroid gland, parathyroid gland, prostate gland or sweat gland.

In another embodiment, assessing the effect of a Clostridial toxin by recording blood flow or a thermal image from a surface of a target site. In an aspect of this embodiment, a Clostridial toxin effect is assessed by recording blood flow or a thermal image of a skin surface of a target site. In another aspect of this embodiment, a Clostridial toxin effect is assessed by recording blood flow or a thermal image of a muscle surface of a target site. In yet another aspect of this embodiment, a Clostridial toxin effect is assessed by recording blood flow or a thermal image of an organ surface of a target site. In still another aspect of this embodiment, a Clostridial toxin effect is assessed by recording blood flow or a thermal image of a gland surface of a target site.

Aspects of the present invention provide, in part, recording blood flow or a thermal image after administration of a Clostridial toxin. As used herein, the term “after” means any length of time following the actual administration of a Clostridial toxin to a mammal. Thus aspects of this embodiment include recording blood flow or a thermal image, e.g., at least one minute after administration of a Clostridial toxin, at least 5 minutes after administration of a Clostridial toxin, at least 15 minutes after administration of a Clostridial toxin, at least 30 minutes after administration of a Clostridial toxin, at least 45 minutes after administration of a Clostridial toxin or at least 60 minutes after administration of a Clostridial toxin. Other aspects of this embodiment include recording blood flow or a thermal image, e.g., at least one hour after administration of a Clostridial toxin, at least two hours after administration of a Clostridial toxin, at least four hours after administration of a Clostridial toxin, at least eight hours after administration of a Clostridial toxin, at least 12 hours after administration of a Clostridial toxin or at least 24 hours after administration of a Clostridial toxin. Further aspects of this embodiment include recording blood flow or a thermal image, e.g., at least one day after administration of a Clostridial toxin, at least two days after administration of a Clostridial toxin, at least four days after administration of a Clostridial toxin, at least eight days after administration of a Clostridial toxin, at least 15 days after administration of a Clostridial toxin or at least 30 days after administration of a Clostridial toxin.

Additional aspects of this embodiment include recording blood flow or a thermal image, e.g., at most one minute after administration of a Clostridial toxin, at most 5 minutes after administration of a Clostridial toxin, at most 15 minutes after administration of a Clostridial toxin, at most 30 minutes after administration of a Clostridial toxin, at most 45 minutes after administration of a Clostridial toxin or at most 60 minutes after administration of a Clostridial toxin. Still other aspects of this embodiment include recording blood flow or a thermal image, e.g., at most one hour after administration of a Clostridial toxin, at most two hours after administration of a Clostridial toxin, at most four hours after administration of a Clostridial toxin, at most eight hours after administration of a Clostridial toxin, at most 12 hours after administration of a Clostridial toxin or at most 24 hours after administration of a Clostridial toxin. Still further aspects of this embodiment include recording blood flow or a thermal image, e.g., at most one day after administration of a Clostridial toxin, at most two days after administration of a Clostridial toxin, at most four days after administration of a Clostridial toxin, at most eight days after administration of a Clostridial toxin, at most 15 days after administration of a Clostridial toxin or at most 30 days after administration of a Clostridial toxin.

Aspects of the present invention provide, in part, comparing one blood flow with another blood flow. As used herein, the term “comparing” means detecting blood flow between two or more different regions at the same time or detecting a blood flow variation of the same region from two or more time points. Thus, in aspects of this embodiment, comparing a first blood flow with a second blood flow can involve, e.g., comparing two or more target sites, comparing two or more non-target sites, comparing a target site to a non-target site, comparing a target site before administration of a Clostridial toxin to the same target site after administration of a Clostridial toxin, comparing a non-target site before administration of a Clostridial toxin to a target site to the same non-target site after administration of a Clostridial toxin to that target site, comparing two or more target site before administration of a Clostridial toxin to the same two or more target site after administration of a Clostridial toxin or comparing two or more non-target sites before administration of a Clostridial toxin to a target site to the same two or more non-target sites after administration of a Clostridial toxin to that target site.

Aspects of the present invention comprise, in part, determining the blood flow in the target site and non-target site after a treatment or challenge. In aspects of this embodiment, determining the blood flow in the target site and non-target site can occur, e.g., at least one second after the treatment or challenge, at least 30 seconds after the treatment or challenge, at least one minute after the treatment or challenge, at least five minutes after the treatment or challenge or at least 15 minutes after the treatment or challenge. In other aspects of this embodiment, determining the blood flow in the target site and non-target site can occur, e.g., at most one minute after the treatment or challenge, at most five minutes after the treatment or challenge, at most 30 minutes after the treatment or challenge, at most 60 minutes after the treatment or challenge or at most 120 minutes after the treatment or challenge.

In one embodiment, a lower blood flow in the first target site as compared to the second target site is indicative of Clostridial toxin activity. In aspects of this embodiment, the blood flow (flux) of a first target site can be, e.g., at least 25% lower than the blood flow (flux) of a second target site, at least 50% lower than the blood flow (flux) of a second target site, at least 75% lower than the blood flow (flux) of a second target site, or at least 100% lower than the blood flow (flux) of a second target site. In other aspects of this embodiment, the blood flow (flux) of a first target site can be, e.g., at least one-fold lower than the blood flow (flux) of a second target site, at least five-fold lower than the blood flow (flux) of a second target site, at least ten-fold lower than the blood flow (flux) of a second target site, or at least one hundred-fold lower than the blood flow (flux) of a second target site. In yet other aspects of this embodiment, the blood flow (flux) of a first target site can be, e.g., at most 25% lower than the blood flow (flux) of a second target site, at most 50% lower than the blood flow (flux) of a second target site, at most 75% lower than the blood flow (flux) of a second target site, at most 100% lower than the blood flow (flux) of a second target site. In yet other aspects of this embodiment, the blood flow (flux) of a first target site can be, e.g., at most one-fold lower than the blood flow (flux) of a second target site, at most five-fold lower than the blood flow (flux) of a second target site, at most ten-fold lower than the blood flow (flux) of a second target site or at most one hundred-fold lower than the blood flow (flux) of a second target site.

In another embodiment, a higher blood flow in the first target site as compared to the second target site is indicative of Clostridial toxin activity. In aspects of this embodiment, the blood flow (flux) of a first target site can be, e.g., at least 25% higher than the blood flow (flux) of a second target site, at least 50% higher than the blood flow (flux) of a second target site, at least 75% higher than the blood flow (flux) of a second target site, or at least 100% higher than the blood flow (flux) of a second target site. In other aspects of this embodiment, the blood flow (flux) of a first target site can be, e.g., at least one-fold higher than the blood flow (flux) of a second target site, at least five-fold higher than the blood flow (flux) of a second target site, at least ten-fold higher than the blood flow (flux) of a second target site, or at least one hundred-fold higher than the blood flow (flux) of a second target site. In yet other aspects of this embodiment, the blood flow (flux) of a first target site can be, e.g., at most 25% higher than the blood flow (flux) of a second target site, at most 50% higher than the blood flow (flux) of a second target site, at most 75% higher than the blood flow (flux) of a second target site, at most 100% higher than the blood flow (flux) of a second target site. In yet other aspects of this embodiment, the blood flow (flux) of a first target site can be, e.g., at most one-fold higher than the blood flow (flux) of a second target site, at most five-fold higher than the blood flow (flux) of a second target site, at most ten-fold higher than the blood flow (flux) of a second target site or at most one hundred-fold higher than the blood flow (flux) of a second target site.

In yet another embodiment, a lower blood flow in the first non-non-target site as compared to the second non-non-target site is indicative of Clostridial toxin activity. In aspects of this embodiment, the blood flow (flux) of a first non-non-target site can be, e.g., at least 25% lower than the blood flow (flux) of a second non-non-target site, at least 50% lower than the blood flow (flux) of a second non-non-target site, at least 75% lower than the blood flow (flux) of a second non-non-target site, or at least 100% lower than the blood flow (flux) of a second non-non-target site. In other aspects of this embodiment, the blood flow (flux) of a first non-non-target site can be, e.g., at least one-fold lower than the blood flow (flux) of a second non-non-target site, at least five-fold lower than the blood flow (flux) of a second non-non-target site, at least ten-fold lower than the blood flow (flux) of a second non-non-target site, or at least one hundred-fold lower than the blood flow (flux) of a second non-target site. In yet other aspects of this embodiment, the blood flow (flux) of a first non-target site can be, e.g., at most 25% lower than the blood flow (flux) of a second non-target site, at most 50% lower than the blood flow (flux) of a second non-target site, at most 75% lower than the blood flow (flux) of a second non-target site, at most 100% lower than the blood flow (flux) of a second non-target site. In yet other aspects of this embodiment, the blood flow (flux) of a first non-target site can be, e.g., at most one-fold lower than the blood flow (flux) of a second non-target site, at most five-fold lower than the blood flow (flux) of a second non-target site, at most ten-fold lower than the blood flow (flux) of a second non-target site or at most one hundred-fold lower than the blood flow (flux) of a second non-target site.

In yet another embodiment, a higher blood flow in the first non-non-target site as compared to the second non-non-target site is indicative of Clostridial toxin activity. In aspects of this embodiment, the blood flow (flux) of a first non-non-target site can be, e.g., at least 25% higher than the blood flow (flux) of a second non-non-target site, at least 50% higher than the blood flow (flux) of a second non-non-target site, at least 75% higher than the blood flow (flux) of a second non-non-target site, or at least 100% higher than the blood flow (flux) of a second non-non-target site. In other aspects of this embodiment, the blood flow (flux) of a first non-non-target site can be, e.g., at least one-fold higher than the blood flow (flux) of a second non-non-target site, at least five-fold higher than the blood flow (flux) of a second non-non-target site, at least ten-fold higher than the blood flow (flux) of a second non-non-target site, or at least one hundred-fold higher than the blood flow (flux) of a second non-target site. In yet other aspects of this embodiment, the blood flow (flux) of a first non-target site can be, e.g., at most 25% higher than the blood flow (flux) of a second non-target site, at most 50% higher than the blood flow (flux) of a second non-target site, at most 75% higher than the blood flow (flux) of a second non-target site, at most 100% higher than the blood flow (flux) of a second non-target site. In yet other aspects of this embodiment, the blood flow (flux) of a first non-target site can be, e.g., at most one-fold higher than the blood flow (flux) of a second non-target site, at most five-fold higher than the blood flow (flux) of a second non-target site, at most ten-fold higher than the blood flow (flux) of a second non-target site or at most one hundred-fold higher than the blood flow (flux) of a second non-target site.

In still another embodiment, a lower blood flow in the target site as compared to the non-target site is indicative of Clostridial toxin activity. In aspects of this embodiment, the blood flow (flux) of a target site can be, e.g., at least 25% lower than the blood flow (flux) of a non-target site, at least 50% lower than the blood flow (flux) of a non-target site, at least 75% lower than the blood flow (flux) of a non-target site, or at least 100% lower than the blood flow (flux) of a non-target site. In other aspects of this embodiment, the blood flow (flux) of a target site can be, e.g., at least one-fold lower than the blood flow (flux) of a non-target site, at least five-fold lower than the blood flow (flux) of a non-target site, at least ten-fold lower than the blood flow (flux) of a non-target site, or at least one hundred-fold lower than the blood flow (flux) of a non-target site. In yet other aspects of this embodiment, the blood flow (flux) of a target site can be, e.g., at most 25% lower than the blood flow (flux) of a non-target site, at most 50% lower than the blood flow (flux) of a non-target site, at most 75% lower than the blood flow (flux) of a non-target site, at most 100% lower than the blood flow (flux) of a non-target site. In yet other aspects of this embodiment, the blood flow (flux) of a target site can be, e.g., at most one-fold lower than the blood flow (flux) of a non-target site, at most five-fold lower than the blood flow (flux) of a non-target site, at most ten-fold lower than the blood flow (flux) of a non-target site or at most one hundred-fold lower than the blood flow (flux) of a non-target site.

In still another embodiment, a higher blood flow in the target site as compared to the non-target site is indicative of Clostridial toxin activity. In aspects of this embodiment, the blood flow (flux) of a target site can be, e.g., at least 25% higher than the blood flow (flux) of a non-target site, at least 50% higher than the blood flow (flux) of a non-target site, at least 75% higher than the blood flow (flux) of a non-target site, or at least 100% higher than the blood flow (flux) of a non-target site. In other aspects of this embodiment, the blood flow (flux) of a target site can be, e.g., at least one-fold higher than the blood flow (flux) of a non-target site, at least five-fold higher than the blood flow (flux) of a non-target site, at least ten-fold higher than the blood flow (flux) of a non-target site, or at least one hundred-fold higher than the blood flow (flux) of a non-target site. In yet other aspects of this embodiment, the blood flow (flux) of a target site can be, e.g., at most 25% higher than the blood flow (flux) of a non-target site, at most 50% higher than the blood flow (flux) of a non-target site, at most 75% higher than the blood flow (flux) of a non-target site, at most 100% higher than the blood flow (flux) of a non-target site. In yet other aspects of this embodiment, the blood flow (flux) of a target site can be, e.g., at most one-fold higher than the blood flow (flux) of a non-target site, at most five-fold higher than the blood flow (flux) of a non-target site, at most ten-fold higher than the blood flow (flux) of a non-target site or at most one hundred-fold higher than the blood flow flux of a non-target site.

Aspects of the present invention provide, in part, comparing a thermal image with another thermal image. As used herein, the term “comparing” means detecting a thermal variation between two or more different regions on a single thermal image or detecting a thermal variation of the same region from two or more thermal images. Thus, in aspects of this embodiment, comparing a thermal image with another thermal image can involve, e.g., comparing two or more target sites, comparing two or more non-target sites, comparing a target site to a non-target site, comparing a target site before administration of a Clostridial toxin to the same target site after administration of a Clostridial toxin, comparing a non-target site before administration of a Clostridial toxin to a target site to the same non-target site after administration of a Clostridial toxin to that target site, comparing two or more target site before administration of a Clostridial toxin to the same two or more target site after administration of a Clostridial toxin or comparing two or more non-target sites before administration of a Clostridial toxin to a target site to the same two or more non-target sites after administration of a Clostridial toxin to that target site.

Aspects of the present invention comprise, in part, determining a thermal image in the target site and non-target site after a treatment or challenge. In aspects of this embodiment, determining a thermal image in the target site and non-target site can occur, e.g., at least one second after the treatment or challenge, at least 30 seconds after the treatment or challenge, at least one minute after the treatment or challenge, at least five minutes after the treatment or challenge or at least 15 minutes after the treatment or challenge. In other aspects of this embodiment, determining a thermal image in the target site and non-target site can occur, e.g., at most one minute after the treatment or challenge, at most five minutes after the treatment or challenge, at most 30 minutes after the treatment or challenge, at most 60 minutes after the treatment or challenge or at most 120 minutes after the treatment or challenge.

Comparing a thermal image with another thermal image can be qualitative or quantitative. Qualitative comparisons can involve visual assessment of images by one skilled in the art to detect thermal variations, such as, e.g., hot or cold spot thermal variations and symmetrical or asymmetrical thermal variations. Quantitative comparisons can involve automated or semi-automated computerized assessment of images to detect thermal variations. As non-limiting examples, BTHERM and CTHERM are open systems for capturing, storing, retrieving and manipulating sequences of thermal images, see, e.g., Bryan F Jones and Peter Plassmann, Digital Infrared Thermal Imaging of Human Skin, 21(6). IEEE Eng. Med. Biol. Mag. 41-48 (2002). Thus in aspects of this embodiment, comparing a thermal image with another thermal image involves detecting a thermal variation of, e.g., at least 0.025° C., at least 0.05° C., at least 0.075° C., at least 0.1° C., at least 0.25° C., at least 0.5° C., at least 0.75° C., at least 1.0° C., at least 2.0° C. or at least 5.0° C. In other aspects of this embodiment, comparing a thermal image with another thermal image involves detecting a thermal variation of, e.g., at most 0.025° C., at most 0.05° C., at most 0.075° C., at most 0.1° C., at most 0.25° C., at most 0.5° C., at most 0.75° C., at most 1.0° C., at most 2.0° C. or at most 5.0° C.

The magnitude of thermal energy variation detected by comparing a thermal image with another thermal image is proportional to the degree of Clostridial toxin effect. As a non-limiting example, detecting a thermal increase of 1° C. in a target site, obtained by comparing thermal images of that target site before and after toxin administration, is indicative of a greater Clostridial toxin effect than detecting a thermal increase of 0.1° C. in a target site, obtained by comparing thermal images of that target site before and after toxin administration. Likewise, detecting a thermal decrease of 1° C. in a target site, obtained by comparing thermal images of that target site before and after toxin administration, is indicative of a greater Clostridial toxin effect than detecting a thermal decrease of 0.1° C. in a target site, obtained by comparing thermal images of that target site before and after toxin administration.

Aspects of the present invention provide, in part, assessing the dispersal of a Clostridial toxin. As used herein, the term “dispersal” means any mode of passive or active transportation of a Clostridial toxin from a target site to a non-target site, including, without limitation, movement by diffusion, movement by passive transport, movement by active transport, movement by the circulatory system, movement by the lymphatic system and movement by retrograde transport.

Aspects of the present invention provide, in part, assessing the dispersal of a Clostridial toxin by recording blood flow or a thermal image from a surface of a target site. Non-limiting examples of a surface include, e.g., a skin surface, a muscle surface, an organ surface or a gland surface. Non-limiting examples of a target site being assessed for dispersal of a Clostridial toxin can include muscle, such as, e.g., skeletal or striated muscle, smooth muscle like visceral muscle and vascular muscle and cardiac muscle; skin, such as, e.g., epidermis, dermis and subdermis; and organs, such as, e.g., bladder, brain, stomach, pancreas, colon, uterus, thyroid gland, parathyroid gland, prostate gland and sweat glands. assessing dispersal of a Clostridial toxin from a target site

Thus, in an embodiment, the dispersal of a Clostridial toxin is assessed by recording blood flow or a thermal image from a surface of a target site. In aspects of this embodiment, a target site being assessed for dispersal of a Clostridial toxin can be, e.g., a muscle, a skin region, an organ or a gland. In further aspects of this embodiment, a target muscle site being assessed for dispersal of a Clostridial toxin can be, e.g., a skeletal muscle, a smooth muscle or a cardiac muscle. In yet further aspects of this embodiment, target skin site being assessed for dispersal of a Clostridial toxin can be, e.g., epidermal skin, dermal skin, subdermal skin and cutaneous skin or subcutaneous skin. In still further aspects of this embodiment, a target organ or gland site being assessed for dispersal of a Clostridial toxin can be, e.g., bladder, brain, stomach, pancreas, colon, uterus, thyroid gland, parathyroid gland, prostate gland or sweat gland.

In another embodiment, the dispersal of a Clostridial toxin is assessed by recording blood flow or a thermal image from a surface of a target site. In an aspect of this embodiment, the dispersal of a Clostridial toxin is assessed by recording blood flow or a thermal image of a skin surface of a target site. In another aspect of this embodiment, the dispersal of a Clostridial toxin is assessed by recording blood flow or a thermal image of a muscle surface of a target site. In yet another aspect of this embodiment, the dispersal of a Clostridial toxin is assessed by recording blood flow or a thermal image of an organ surface of a target site. In still another aspect of this embodiment, the dispersal of a Clostridial toxin is assessed by recording blood flow or a thermal image of a gland surface of a target site.

Aspects of the present invention provide, in part, assessing the dispersal of a Clostridial toxin by recording blood flow or a thermal image from a surface of a non-target site. As used herein, the term “non-target site” means a particular area of a mammalian body for which administration of a Clostridial toxin is not being considered or is undesired. Non-limiting examples of a non-target site being assessed for dispersal of a Clostridial toxin can include muscle, such as, e.g., skeletal or striated muscle, smooth muscle like visceral muscle and vascular muscle and cardiac muscle; skin, such as, e.g., epidermal skin, dermal skin, subdermal skin and cutaneous skin and subcutaneous skin; and organs, such as, e.g., bladder, brain, stomach, pancreas, colon, uterus, thyroid gland, parathyroid gland, prostate gland and sweat glands. Non-limiting examples of a surface that a thermal image can be recorded can include, e.g., a skin surface, a muscle surface, an organ surface or a gland surface. Generally, administration of a Clostridial toxin is well tolerated. However, the administered toxin may diffuse to areas other than the target site, namely a non-target site, particularly when high toxin doses are administered. For example, a patient administered a therapeutically effective amount MyoBloc™/NeuroBloc™ into the neck muscles for torticollis may develop dysphagia because of dispersal of the toxin into the oropharynx.

Thus, in an embodiment a non-target site is assessed for dispersal of a Clostridial toxin from a target site. In aspects of this embodiment, a non-target site assessed for dispersal of a Clostridial toxin can be, e.g., a muscle, a skin region, an organ or a gland. In further aspects of this embodiment, a non-target muscle site being assessed for dispersal of a Clostridial toxin can be, e.g., a skeletal muscle, a smooth muscle or a cardiac muscle. In yet further aspects of this embodiment, non-target skin site being assessed for dispersal of a Clostridial toxin can be, e.g., epidermal skin, dermal skin, subdermal skin and cutaneous skin or subcutaneous skin. In still further aspects of this embodiment, an non-target organ or gland site being assessed for dispersal of a Clostridial toxin can be, e.g., bladder, brain, stomach, pancreas, colon, uterus, thyroid gland, parathyroid gland, prostate gland or sweat gland.

In another embodiment, the dispersal of a Clostridial toxin is assessed by recording blood flow or a thermal image from a surface of a non-target site. In an aspect of this embodiment, the dispersal of a Clostridial toxin is assessed by recording blood flow or a thermal image of a skin surface of a non-target site. In another aspect of this embodiment, the dispersal of a Clostridial toxin is assessed by recording blood flow or a thermal image of a muscle surface of a non-target site. In yet another aspect of this embodiment, the dispersal of a Clostridial toxin is assessed by recording blood flow or a thermal image of an organ surface of a non-target site. In still another aspect of this embodiment, the dispersal of a Clostridial toxin is assessed by recording blood flow or a thermal image of a gland surface of a non-target site.

It is envisioned that dispersal of toxin from a target site to a non-target site can be detected at any and all distances according to the methods disclosed in the present specification, with the proviso that the dispersal distance is within the range of detection sensitivity of the method used. The dispersal distance of a Clostridial toxin can be evaluated locally, e.g., by assessing the toxin's effect in the non-target sites immediately surrounding the target site, or systemically, e.g., by assessing the toxin's effect in the non-target sites not nearby the target site, such as, e.g., a region proximal to the target site, a region distal to the target site, a region ipsilateral to the target site. or a region contralateral to the target site. The dispersal distance of a Clostridial toxin can be evaluated within the same muscle, skin region, organ or gland, or the dispersal distance of a Clostridial toxin can be evaluated between two or more different muscles, skin regions, organs or glands.

Thus, in one embodiment, the dispersal of a Clostridial toxin can be detected in non-target sites immediately surrounding the target site. In another embodiment, the dispersal of a Clostridial toxin can be detected in non-target sites not nearby the target site. In yet another embodiment, the dispersal of a Clostridial toxin can be detected locally. In yet another embodiment, the dispersal of a Clostridial toxin can be detected systemically. In aspects of this embodiment, the dispersal of a Clostridial toxin can be detected in a non-target site at a distance of, e.g., at most 0.1 cm from the target site, at most 0.5 cm from the target site, at most 1.0 cm from the target site, at most 5.0 cm from the target site, at most 10 cm from the target site, at most 50 cm from the target site, at most 100 cm from the target site and at most 150 cm from the target site. In other aspects of this embodiment, the dispersal of a Clostridial toxin can be detected in a non-target site at a distance of, e.g., at least 0.1 cm from the target site, at least 0.5 cm from the target site, at least 1.0 cm from the target site, at least 5.0 cm from the target site, at least 10 cm from the target site, at least 50 cm from the target site, at least 100 cm from the target site and at least 150 cm from the target site.

Comparing a thermal image with another thermal image can be qualitative or quantitative. Qualitative comparisons can involve visual assessment of images by one skilled in the art to detect thermal variations, such as, e.g., hot or cold spot thermal variations and symmetrical or asymmetrical thermal variations. Quantitative comparisons can involve automated or semi-automated computerized assessment of images to detect thermal variations. As non-limiting examples, BTHERM and CTHERM are open systems for capturing, storing, retrieving and manipulating sequences of thermal images, see, e.g., Bryan F Jones and Peter Plassmann, Digital Infrared Thermal Imaging of Human Skin, 21(6). IEEE Eng. Med. Biol. Mag. 41-48 (2002). Thus in aspects of this embodiment, comparing a thermal image with another thermal image involves detecting a thermal variation of, e.g., at least 0.025° C., at least 0.05° C., at least 0.075° C., at least 0.1° C., at least 0.25° C., at least 0.5° C., at least 0.75° C., at least 1.0° C., at least 2.0° C. or at least 5.0° C. In other aspects of this embodiment, comparing a thermal image with another thermal image involves detecting a thermal variation of, e.g., at most 0.025° C., at most 0.05° C., at most 0.075° C., at most 0.1° C., at most 0.25° C., at most 0.5° C., at most 0.75° C., at most 1.0° C., at most 2.0° C. or at most 5.0° C.

The magnitude of thermal energy variation detected by comparing a thermal image with another thermal image is proportional to the degree of Clostridial toxin dispersal. As a non-limiting example, detecting a thermal increase of 1° C. in a non-target site, obtained by comparing thermal images of that non-target site before and after toxin administration, is indicative of greater Clostridial toxin dispersal than a detecting a thermal increase of 0.1° C. in a non-target site, obtained by comparing thermal images of that non-target site before and after toxin administration. Likewise, detecting a thermal decrease of 1° C. in a non-target site, obtained by comparing thermal images of that non-target site before and after toxin administration, is indicative of a greater Clostridial toxin dispersal than detecting a thermal decrease of 0.1° C. in a non-target site, obtained by comparing thermal images of that non-target site before and after toxin administration.

It is envisioned that any of the disclosed methods of the present specification pertaining to blood flow can be used in conjunction with any of the disclosed methods of the present specification pertaining to heat dissipation. For example, laser-Doppler flowmetry can be used to assess superficial blood flow, whereas thermal imaging reflects a local warming reaction depending, in part, on blood flow in subcutaneous tissues. As such, measuring vascular changes by both methods provides information on the vasomotor status of both superficial and deeper skin layers.

Aspects of the present invention can also be described as follow:
1. A method of assessing a physiological activity of a target site for administration of a Clostridial toxin to a mammal, the method comprising the step of recording a thermal image from a surface of the target site in the mammal prior to a Clostridial toxin administration.
2. The method according to 1, wherein the recording is taken under resting conditions.
3. The method according to 1, wherein the recording is taken under non-resting conditions.
4. The method according to 1, wherein the surface comprises a muscle surface, a skin surface, an organ surface or a gland surface.
5. The method according to 1, wherein the target site comprises a muscle, a skin region, an organ or a gland.
6. The method according to 1, wherein the mammal consists of a rodent, a rabbit, a porcine, a bovine, an equine, a non-human primate or a human.
7. A method of administering a Clostridial toxin to a target site in a mammal, the method comprising the steps of:
   a. recording a thermal image from a surface of the target site in the mammal prior to a Clostridial toxin administration; and
   b. administering the Clostridial toxin to the target site.
8. The method according to 7, wherein the recording is taken under resting conditions.
9. The method according to 7, wherein the recording is taken under non-resting conditions.
10. The method according to 7, wherein the surface comprises a muscle surface, a skin surface, an organ surface or a gland surface.
11. The method according to 7, wherein the target site comprises a muscle, a skin region, an organ or a gland.
12. The method according to 7, wherein the mammal consists of a rodent, a rabbit, a porcine, a bovine, an equine, a non-human primate or a human.
13. The method according to 7, wherein administering the Clostridial toxin is by injection.
14. A method of assessing an effect of a Clostridial toxin on a target site in a mammal, the method comprising the steps of:
   a. recording a first thermal image from a surface of the target site in the mammal prior to administration of the Clostridial toxin;
   b. recording a second thermal image from the surface of the target site in the mammal after the administration of the Clostridial toxin; and
   c. comparing the first thermal image recording of step (a) to the second thermal image recording of step (b);
      wherein a difference between the first thermal image recording and the second thermal image recording is indicative of a Clostridial toxin effect.
15. The method according to 14, wherein the first thermal image recording is taken under resting conditions.
16. The method according to 14, wherein the first thermal image recording is taken under non-resting conditions.
17. The method according to 14, wherein the second thermal image recording is taken under resting conditions.
18. The method according to 14, wherein the second thermal image recording is taken under non-resting conditions.
19. The method according to 14, wherein the surface comprises a muscle surface, a skin surface, an organ surface or a gland surface.
20. The method according to 14, wherein the target site comprises a muscle, a skin region, an organ or a gland.
21. The method according to 14, wherein the mammal consists of a rodent, a rabbit, a porcine, a bovine, an equine, a non-human primate or a human.
22. The method according to 14, wherein administering the Clostridial toxin is by injection.
23. The method according to 14, wherein the comparison of step (c) is qualitative.
24. The method according to 14, wherein the comparison of step (c) is quantitative.
25. A method of assessing dispersal of a Clostridial toxin from a target site to a non-target site in a mammal, the method comprising the steps of:
   a. recording a first thermal image from a surface of the target site in the mammal and a first thermal image from a surface of the non-target site of the mammal prior to administration of the Clostridial toxin;
   b. recording a second thermal image from the surface of the target site in the mammal and a second thermal image from the surface of the non-target site of the mammal after the administration of the Clostridial toxin; and
   c. comparing the first thermal image of the target site and the first thermal image of the non-target site of step (a) to the second thermal image of the target site and the second thermal image of the non-target site of step (b);
      wherein a difference between the first thermal image recording of the non-target site and the second thermal image recording of the non-target site is indicative of dispersal of a Clostridial toxin from a target site.
26. The method according to 25, wherein the first thermal image recording is taken under resting conditions.
27. The method according to 25, wherein the first thermal image recording is taken under non-resting conditions.
28. The method according to 25, wherein the second thermal image recording is taken under resting conditions.
29. The method according to 25, wherein the second thermal image recording is taken under non-resting conditions.
30. The method according to 25, wherein the target site surface comprises a muscle surface, a skin surface, an organ surface or a gland surface.
   31. The method according to 25, wherein the target site comprises a muscle, a skin region, an organ or a gland.
32. The method according to 25, wherein the non-target site surface comprises a muscle surface, a skin surface, an organ surface or a gland surface.
33. The method according to 25, wherein the non-target site comprises a muscle, a skin region, an organ or a gland.
34. The method according to 25, wherein the mammal consists of a rodent, a rabbit, a porcine, a bovine, an equine, a non-human primate or a human.
35. The method according to 25, wherein administering the Clostridial toxin is by injection.
36. The method according to 25, wherein the comparison of step (c) is qualitative.
37. The method according to 25, wherein the comparison of step (c) is quantitative.
38. A method of assessing a physiological activity of a target site for administration of a Clostridial toxin to a mammal, the method comprising the step of recording blood flow from a surface of the target site in the mammal prior to a Clostridial toxin administration.
39. The method according to 38, wherein the recording is taken under resting conditions.
40. The method according to 38, wherein the recording is taken under non-resting conditions.
41. The method according to 38, wherein the surface comprises a muscle surface, a skin surface, an organ surface or a gland surface.
42. The method according to 38, wherein the target site comprises a muscle, a skin region, an organ or a gland.
43. The method according to 38, wherein the mammal consists of a rodent, a rabbit, a porcine, a bovine, an equine, a non-human primate or a human.
44. A method of administering a Clostridial toxin to a target site in a mammal, the method comprising the steps of:
   a. recording blood flow from a surface of the target site in the mammal prior to a Clostridial toxin administration; and
   b. administering the Clostridial toxin to the target site.
45. The method according to 44, wherein the recording is taken under resting conditions.
46. The method according to 44, wherein the recording is taken under non-resting conditions.
47. The method according to 44, wherein the surface comprises a muscle surface, a skin surface, an organ surface or a gland surface.
48. The method according to 44, wherein the target site comprises a muscle, a skin region, an organ or a gland.
49. The method according to 44, wherein the mammal consists of a rodent, a rabbit, a porcine, a bovine, an equine, a non-human primate or a human.
50. The method according to 44, wherein administering the Clostridial toxin is by injection.
51. A method of assessing an effect of a Clostridial toxin on a target site in a mammal, the method comprising the steps of:
   a. recording a first blood flow from a surface of the target site in the mammal prior to administration of the Clostridial toxin;
   b. recording a second blood flow from the surface of the target site in the mammal after the administration of the Clostridial toxin; and
   c. comparing the first blood flow recording of step (a) to the second blood flow recording of step (b).
      wherein a difference between the first blood flow recording and the second blood flow recording is indicative of a Clostridial toxin effect.
52. The method according to 51, wherein the first blood flow recording is taken under resting conditions.
53. The method according to 51, wherein the first blood flow recording is taken under non-resting conditions.
54. The method according to 51, wherein the second blood flow recording is taken under resting conditions.
55. The method according to 51, wherein the second blood flow recording is taken under non-resting conditions.
56. The method according to 51, wherein the surface comprises a muscle surface, a skin surface, an organ surface or a gland surface.
57. The method according to 51, wherein the target site comprises a muscle, a skin region, an organ or a gland.
58. The method according to 51, wherein the mammal consists of a rodent, a rabbit, a porcine, a bovine, an equine, a non-human primate or a human.
59. The method according to 51, wherein administering the Clostridial toxin is by injection.
60. The method according to 51, wherein the comparison of step (c) is qualitative.
61. The method according to 51, wherein the comparison of step (c) is quantitative.
62. The method according to 51, wherein the blood flow is measured by a laser-Doppler technique.
63. The method of 40, wherein the Clostridial toxin is a BoNT/A, a BoNT/B, a BoNT/C1, a BoNT/D, a BoNT/E, a BoNT/F, a BoNT/G or a TeNT.
65. A method of assessing dispersal of a Clostridial toxin from a target site to a non-target site in a mammal, the method comprising the steps of:
   a. recording a first blood flow from a surface of the target site in the mammal and a first blood flow from a surface of the non-target site of the mammal prior to administration of the Clostridial toxin;
   b. recording a second blood flow from the surface of the target site in the mammal and a second blood flow from the surface of the non-target site of the mammal after the administration of the Clostridial toxin; and
   c. comparing the first blood flow recording of the target site and the first blood flow recording of the non-target site of step (a) to the second blood flow recording of the target site and the second blood flow recording of the non-target site of step (b).
      wherein a difference between the first blood flow recording of the non-target site and the second blood flow recording of the non-target site is indicative of dispersal of a Clostridial toxin from a target site.
66. The method according to 65, wherein the first blood flow recording is taken under resting conditions.
67. The method according to 65, wherein the first blood flow recording is taken under non-resting conditions.
68. The method according to 65, wherein the second blood flow recording is taken under resting conditions.
69. The method according to 65, wherein the second blood flow recording is taken under non-resting conditions.
70. The method according to 65, wherein the target site surface comprises a muscle surface, a skin surface, an organ surface or a gland surface.
71. The method according to 65, wherein the target site comprises a muscle, a skin region, an organ or a gland.
72. The method according to 65, wherein the non-target site surface comprises a muscle surface, a skin surface, an organ surface or a gland surface.
73. The method according to 65, wherein the non-target site comprises a muscle, a skin region, an organ or a gland.
74. The method according to 65, wherein the mammal consists of a rodent, a rabbit, a porcine, a bovine, an equine, a non-human primate or a human.
75. The method according to 65, wherein administering the Clostridial toxin is by injection.
76. The method according to 65, wherein the comparison of step (c) is qualitative.
77. The method according to 65, wherein the comparison of step (c) is quantitative.
78. The method according to 65, wherein the blood flow is measured by a laser-Doppler technique.
79. The method according to 65, wherein the Clostridial toxin is a BoNT/A, a BoNT/B, a BoNT/C1, a BoNT/D, a BoNT/E, a BoNT/F, a BoNT/G or a TeNT.
80. A method of identifying a neurogenic inflammation inhibitor, the method comprising the steps of:
   a) administering to a mammal an effective amount of a neurogenic inflammation inhibitor to a target site and a control treatment to a non-target site;
   b) administering an effective amount of a challenger to the target site and to the non-target site, wherein the challenger is administered after the administration of the neurogenic inflammation inhibitor; and
   c) recording the blood flow in the target site and non-target site, where a lower blood flow in the target site as compared to the non-target site is indicative of a neurogenic inflammation inhibitor.
81. The method according to 80, wherein the blood flow is measured by a laser-Doppler technique.
82. The method according to 80, wherein the challenger is an Aα-fiber agonist, an Aβ-fiber agonist, an Aγ-fiber agonist, an Aδ-fiber agonist, or a C-fiber agonist
83. A method of assessing an inhibitory effect of a Clostridial toxin on neurogenic inflammation in a target site of a mammal, the method comprising the steps of:
   a) administering to a mammal an effective amount of a Clostridial toxin to a target site and a control treatment to a non-target site;
   b) administering an effective amount of a challenger to the target site and to the non-target site, wherein the challenger is administered after the administration of the Clostridial toxin; and
   c) recording the blood flow in the target site and non-target site;
      wherein a lower blood flow in the target site as compared to the non-target site is indicative of a Clostridial toxin effect on neurogenic inflammation.
84. The method according to 83, wherein the blood flow is measured by a laser-Doppler technique.
85. The method according to 83, wherein the Clostridial toxin is a BoNT/A, a BoNT/B, a BoNT/C1, a BoNT/D, a BoNT/E, a BoNT/F, a BoNT/G or a TeNT.
86. The method according to 83, wherein the challenger is an Aα-fiber agonist, an Aβ-fiber agonist, an Aγ-fiber agonist, an Aδ-fiber agonist, or a C-fiber agonist

### EXAMPLES

The following non-limiting examples are provided for illustrative purposes only in order to facilitate a more complete understanding of disclosed embodiments and are in no way intended to limit any of the embodiments disclosed in the present invention.

### Example 1

### Assessing a Physiological Activity of a Target Site for a Clostridial Toxin Administration Using Heat Dissipation

This example illustrates how examining heat dissipation can identify a target site for administering a Clostridial toxin.

A 44 year old male patient suffers from intense pain due to a task-specific dystonia affecting the palm and fingers of the right hand. To determine the location of the dystonic areas as well as to assess whether a Clostridial toxin administration would be appropriate for treating this affliction, the physician employs thermal imaging technique to assess the physiological activity of the man's palm and fingers of the right hand.

The male patient is prepared for thermal imaging under resting conditions. The male patient is prepared for thermal imaging under resting conditions. This is done by asking the patient to disrobe the affected area and letting the patient lie down in a supine position in a climate controlled room held at 20±1° C. for a period of approximately 25 minutes. The scanner unit is positioned at a distance of approximately 20 cm perpendicular to the affected hand, thereby allowing maximum coverage of the target site. A thermal image of the hand is then taken and the digital image is stored on a computer hard drive. One thermographic imaging system that may be used in accordance with aspects of the present invention is the Agema Thermovision 900 series (AGEMA Infrared Systems AB, Danderyd, Sweden). The scanner is a long-wave, cryogenically cooled system utilizing a mercury cadmium telluride detector with a spectral response of 8-12 um and a sensitivity of 0.1° C. at 30° C. This window of 8-12 um coincides with the region of maximal skin emission of 8-10 um. The scanner is controlled by a dedicated system controller which runs software specifically for thermal image analysis. After visual examination of the thermal image, the physician determines that three muscle groups show a dramatically increase in thermal energy being emitted from the affected hand as compared to the male patient's unaffected left hand and with thermal images of hands that are taken from other patients unaffected by task-specific dystonia. The physician recommends administering a Clostridial toxin to the muscle groups showing increased thermal energy emittance to alleviate the task-specific dystonia.

### Example 2

### Administering a Clostridial Toxin to a Target Site Using Heat Dissipation

This example illustrates how examining heat dissipation will provide information regarding where to administer a Clostridial toxin to a target site.

A 38 year old female patient presents with a severe case of axillary hyperhidrosis. The treating physician recommends administering a botulinum toxin type A to the affected areas of hyperhidrosis. To determine which sweat glands to treat, the physician employs a thermal imaging technique to assess the physiological activity of the axillary areas undergoing excessive sweating.

The female patient is prepared for thermal imaging under resting conditions. This is done by asking the patient to disrobe the affected area and letting the patient lie down in a supine position in a climate controlled room held at 20±1° C. for a period of approximately 15 minutes. Once the patient becomes acclimated to the environment, she is then seated in a dental chair. The scanner unit is positioned at a distance of approximately 50 cm perpendicular to the affected axillary area in order to achieve maximum coverage of the target site. A thermal image of the area is then taken using, e.g., the thermographic imaging system described in Example 1, and the digital image is stored on a computer hard drive. Computer analysis of the thermal image reveals five target sites encompassing an 8×15 cm² region that exhibit a statistically significant increase in the thermal energy being emitted from the affected areas of hyperhidrosis in the female patient as compared to other patients unaffected by axillary hyperhidrosis.

The physician then proceeds to administer a Clostridial toxin to the areas showing increased thermal energy emittance to alleviate the excessive sweating. Based on the thermal image, the target sites are mapped within the 8×15 cm² region. Crystal ice particle coated with Botulinum toxin type A are loaded into a needleless injector. The projection pressure is set so that the drug particles may be delivered to the dermis layer of the skin. Also, the amount of the drug particle is loaded so that approximately 20 units to approximately 60 units of botulinum toxin type A is delivered to the five target sites within the 8×15 cm² region.

### Example 3

### Assessing the Effect of a Clostridial Toxin Administration Using Heat Dissipation

This example illustrates how comparing a thermal image of a target site before and after the administration of a Clostridial toxin will provide information regarding the degree of a Clostridial toxin effect on that target site.

A 53 year old female patient suffers from intense pain due to a temporomandibular joint dysfunction. The treating physician recommends administering 10 units of botulinum toxin type A to her masseter muscles to alleviate the pain. To determine the muscle sites to treat, the physician employs a thermal imaging technique to assess the physiological activity of the area undergoing intense pain.

The female patient is prepared for thermal imaging under resting conditions. This is done by asking the patient to disrobe the affected area and letting the patient lie down in a supine position in a climate controlled room held at 20±1° C. for a period of approximately 15 minutes. In addition, all facial cosmetics are removed and the skin surface allowed to air dry. Hair is held back off the face with hair clips and a reflective marker is placed on the skin overlying the anterior edge of the masseter muscle. Once the patient becomes acclimated to the environment, she is then seated in a dental chair. The scanner unit is positioned at a distance of approximately 30 cm perpendicular to the affected temporomandibular joint area in order to achieve maximum coverage of the target site. Thermal images of both sides of the face are then taken using, e.g., the thermographic imaging system described in Example 1, and the digital images are stored on a computer hard drive. Computer analysis of the thermal image reveals that the masseter muscle on both sides of the face exhibit a statistically significant increase in the thermal energy being emitted from the affected experiencing pain in the female patient as compared to other patients unaffected by temporomandibular joint dysfunction.

The physician then proceeds to administer a Clostridial toxin to the areas showing increased thermal energy emittance to alleviate the pain. Based on the thermal image, the target sites within the masseter muscle are mapped and the physician administers approximately 10 units of botulinum toxin type A to the target sites within the masseter muscles on each side of the patient's face of the patient. The patient is discharged and is asked to return for a second scan in 24 hours. Further, to prevent the unwanted dispersal of botulinum toxin into the adjacent muscles, the patient is instructed to not massage the administration site, and is advised to not reapply her makeup in the office.

The next day, the female patient returns as is prepared for thermal imaging under resting conditions as described above. Thermal images of both sides of the face are then taken using, e.g., the thermographic imaging system described in Example 1, and the digital images are stored on a computer hard drive. The thermographic system includes software that calculates the temperature differences between the first and the second thermal image. The alignment and subtraction of images is undertaken by superimposing two reference markers on each of the images of interest. For greater accuracy, surface reference markers of a highly reflective nature should be placed over recognized anatomical sites prior to the functional test. These markers allow for greater accuracy in the overlay procedure and therefore a more accurate result after pixel subtraction. Comparison of the two images indicate that the muscles administered botulinum toxin show a decrease in temperature which approximates the temperature exhibited by masseter muscles from patients unaffected by temporomandibular joint dysfunction. This comparison also shows that muscles not administered botulinum toxin show a temperature change of approximately 0° C. Upon analysis of these thermal images, the physician determines that additional administration of botulinum toxin is not warranted.

### Example 4

### Assessing the Dispersal of a Clostridial Toxin Administration Using Heat Dissipation

This example illustrates how comparing a thermal image of a target site before and after the administration of a Clostridial toxin will provide information regarding the degree of a Clostridial toxin effect on a target site and any possible dispersal of the toxin away from the target site to a non-target site.

A 33 year old male patient suffers from intense pain due to a muscle spasm in his left calf. The treating physician recommends administering 10 units of botulinum toxin type A to the calf muscle to alleviate the pain. To make sure that the administered botulinum toxin does not diffuse to unintended muscles, the physician employs a thermal imaging technique to assess the physiological activity of the area undergoing intense pain.

The male patient is prepared for thermal imaging under resting conditions. This is done by asking the patient to disrobe the affected area and letting the patient lie down in a supine position in a climate controlled room held at 20±1° C. for a period of approximately 25 minutes. The scanner unit is positioned at a distance of approximately 50 cm perpendicular to the affected leg in order to achieve maximum coverage of both the target and non-target sites. A thermal image of both the affected left calf and the unaffected right calf areas are then taken using, e.g., the thermographic imaging system described in Example 1, and the digital image is stored on a computer hard drive. Computer analysis of the thermal image reveals three target sites that exhibit a statistically significant increase in the thermal energy being emitted from the spasmodic calf area of the male patient as compared to the unaffected right calf area of the male patient as well as other patients not experiencing muscle spasms in the calf.

The physician then proceeds to administer a Clostridial toxin to the areas showing increased thermal energy emittance to alleviate the muscle spasm and associated pain. Based on the thermal image, the target sites within the calf muscles are mapped and the physician administers approximately 10 units of botulinum toxin type A to the target sites within the muscles of the left calf. The patient is discharged and is asked to return for a second scan in 24 hours. Further, to prevent the unwanted dispersal of botulinum toxin into the adjacent muscles, the patient is instructed to not massage the target site and avoid exertion on the day of treatment.

The next day, the male patient returns and is prepared for thermal imaging under resting conditions as described above. Thermal images of the left and right calves are then taken using, e.g., the thermographic imaging system described in Example 1, and the digital images are stored on a computer hard drive. Analysis of temperature differences between the first and the second thermal image are performed, e.g., as described in Example 3. Comparison of the two images indicate that most of the calf muscles administered botulinum toxin show a decrease in temperature which approximates the temperature exhibited by the unaffected right calf muscles from the patient. However, a small region from one of the identified target sites still emits an increased thermal energy, indicating an additional toxin administration is required. In addition, examination of the non-target sites reveal that these sites do not show a change in thermal energy emittence, indicating that the toxin did not diffuse into these non-target sites. Therefore, upon analysis of these thermal images, the physician determines that additional administration of botulinum toxin should be administered in the remaining target site showing a difference in thermal energy relative to the unaffected right calf muscle.

### Example 5

### Assessing the Effective Threshold Toxic Dose of a Clostridial Toxin Administration Using a Systemic Heat Dissipation Assay

This example illustrates how the effective threshold dose of a Clostridial toxin formulation can be determined by assessing the effect of a Clostridial toxin administration in a mammal using heat dissipation.

Currently, the effective lethal dose of a Clostridial toxin is determined using an in vivo assay that measures animal lethality, such as, e.g., the mouse lethality assay (MLA or LD₅₀ assay). The standard LD₅₀ assay evaluates the dose-dependent lethality of toxin preparations. However, the high doses of a Clostridial toxin necessary to achieve lethality also result in a systemic hypothermic response in the animal due to the disruption of many physiological processes that effect thermal regulation. This induced hypothermic response, due to the systemic responses to a Clostridial toxin administration, can be used as a readout of a toxin effect. In addition, Clostridial toxin-mediated changes in the physiological state of a mammal occur well before the onset of lethality. Thus, the detectable thermal energy changes resulting from the milder toxicity of lower non-lethal doses of a toxin can be used as an assay endpoint to determine the threshold systemic effects of a toxin rather than the lethal effects of the toxin. Therefore determining the effective threshold dose of a Clostridial toxin using a thermal imaging assay will greatly reduce the pain and suffering of the animals.

To determine the effective threshold dose of a Clostridial toxin formulation, the effect of a Clostridial toxin administration is assessed using a thermal imaging assay. Mice are prepared for thermal imaging under resting conditions. Mice are then lightly anesthetized with Isoflurane and a thermal image of the ventral thorax region from each mouse is acquired using a TSA ImagIR System (Seahorse Bioscience, North Billerica, Mass.) and the digital image is stored on a computer hard drive. Various doses of a BoNT/A formulation are then administered to the mice following recovery from anesthesia. In a typical assay, a BoNT/A stock solution is used to generate dose dilutions over a dose range of 30-60 U/kg (e.g. 30 U/kg, 36 U/kg, 42 U/kg, 48 U/kg, 54 U/kg, and 60 U/kg), with dilutions made in vehicle (0.5% BSA/saline solution). Dosing is based on the median weight per dose group, with mouse weights ranging from 17 grams to 30 grams, where the weight range of any single dose group of mice is no greater than +/−2 grams, and dose groups consist of 10 mice each. Mice are injected intraperitoneally with either vehicle (control) or the specific toxin dose dilution, delivered via a 27 gauge needle, and each mouse receives a dose volume based on individual weight, such that the desired dose (in U/kg) is delivered in a volume of 10 mL/kg.

The next day, each mouse is prepared for thermal imaging under non-resting conditions as described above. A whole body thermal image of each mouse is then taken using, e.g., the thermographic imaging system described above, and the digital images are stored on a computer hard drive. Analysis of temperature differences between the first and the second thermal image for each dose are performed, e.g., as described in Example 3 and a dose response curve is derived from nonlinear regression analysis of these data, establishing a thermographic dose-response measure of non-lethal toxicity. The dose that results in 50% of the mice exhibiting a statistically significant decrease in the thermal energy being emitted after administration of the BoNT/A preparation as compared to control animals, e.g., the thermal images of the same mice before administration of the BoNT/A preparation or different mice administered a saline control (a normothermic vehicle control group), is the effective threshold dose (TD₅₀).

### Example 6

### Assessing the Effective Threshold Pharmacological Dose of a Clostridial Toxin Administration Using a Local Target Site Heat Dissipation Assay

This example illustrates how the effective threshold dose of a Clostridial toxin formulation can be determined by assessing the effect of a Clostridial toxin administration in a mammal using heat dissipation.

The effective pharmacological dose of a Clostridial toxin is determined using an in vivo assay that measures muscle paralysis, such as, e.g., the Digit Abduction Score (DAS) assay or the Gastrocnemius Paralysis Assay (GPA). Muscle paralysis results in a decrease in the heat output by an exercised muscle due to failure in muscle contraction and the generation of heat. This decrease in induced thermal output following exercise (hypothermia) can be used as a readout of a Clostridial toxin effect. The doses used in pharmacological studies are non-toxic and non lethal and should only elicit paralytic responses in the muscle of injection (e.g. gastrocnemius) or in adjacent muscles as a measure of local intermuscular diffusion (e.g. tibialis anterior, extensor digitorum longus, quadriceps).

To determine the effective pharmacological dose of a Clostridial toxin formulation, the effect of a Clostridial toxin administration is assessed using a thermal imaging assay. Mice are prepared for thermal imaging under non-resting conditions by physical stimulation using a treadmill (15 degree incline @ 5 meters/minute) for 10 minutes. Mice are then lightly anesthetized with Isoflurane and thermal images are acquired of the skin (fur shaved) overlying the right (ipsilateral) and left (contralateral) gastroxcnemius muscles (fur shaved) of each mouse using a TSA ImagIR System (Seahorse Bioscience, North Billerica, Mass.) and the digital image is stored on a computer hard drive. Various doses of a BoNT/A formulation are administered to the mice by intramuscular injection in the tail (five mice/dose). In a typical assay, a BoNT/A stock solution is used to generate dose dilutions over a dose range of 1-60 U/kg, with dilutions made in vehicle (0.5% BSA/saline solution). Dosing is based on the median weight per dose group, with mouse weights ranging from 17 grams to 30 grams, where the weight range of any single dose group of mice is no greater than +/−4 grams, and dose groups consist of 6 mice each. The injection volume is 5 or 10 μL, delivered via a 30 gauge needle using a Hamilton syringe affixed with a ratcheting, volume-adjustable dispenser. BoNT/A solution is injected into the distal portion of the medial head of the right gastrocnemeus muscle, using the posterior medial malleolar groove as a needle guide. Progressive paralysis is then evaluated daily for four days to capture the peak paralytic effects (typically between three to four days post-injection of BoNT/A). Prior to collection of thermal images, mice are similarly exercised on the treadmill to stimulate muscle activity, followed by anesthesia and thermography. Images from test groups are compared (qualitatively and quantitatively) to images from the vehicle control group (normothermic). The degree of hypothermia (net difference from vehicle control) is calculated for each dose administered and a ED₅₀ (the dose producing 50% paralysis) is derived from nonlinear regression analysis of these data, establishing a thermographic dose-response measure of muscle paralysis.

### Example 7

### Assessing Immunoresistance to a Clostridial Toxin Administration Using Heat Dissipation

This example illustrates how immunoresistance to a Clostridial toxin can be determined by assessing the effect of a Clostridial toxin administration in a mammal using a thermal imaging system.

Immunoresistance to a Clostridial toxin in a mammal is usually determined using an in vivo assay that measures animal lethality, such as, e.g., the mouse protection assay (MPA). The current standard MPA evaluates the degree of protection conferred by anti-Clostridial toxin-neutralizing antibodies against a lethal challenge dose of a toxin. However, the high doses of a Clostridial toxin necessary to achieve lethality also result in a systemic hypothermic response in the animal due to the disruption of many physiological processes that effect thermoregulation. This induced hypothermic response, due to the systemic responses to a Clostridial toxin administration, can be used as a readout of a toxin effect. In addition, Clostridial toxin-mediated changes in the physiological state of a mammal occur well before the onset of lethality. Thus, the detectable thermal energy changes resulting from the milder toxicity of lower non-lethal doses of a toxin can be used to infer the presence of toxin-neutralizing antibodies since the presence of toxin-neutralizing antibodies will effectively lower the challenge toxin dose (when combined), producing a graded toxicity response to the otherwise lethal challenge dose. Therefore determining the presence of anti-Clostridial toxin-neutralizing antibodies using a thermal imaging assay will greatly reduce the pain and suffering of the animals.

To determine the immunoresistance of a cervical dystonia patient, the effect of a Clostridial toxin administration is assessed using a thermal imaging assay. First, the maximum toxic dose (LD₉₉) of a BoNT/A preparation is determine, e.g., as described in Example 5, except that the various doses of a BoNT/A formulation are administered to the mice by intravenous injection in the tail. Second, mice are prepared for thermal imaging under resting conditions. Mice are then lightly anesthetized with Isoflurane and a thermal image of the ventral thorax region from each mouse is acquired using a TSA ImagIR System (Seahorse Bioscience, North Billerica, Mass.) and the digital image is stored on a computer hard drive. A blood sample from each patient is processed to obtain the serum. A 100 μL aliquot of serum from each patient is mixed with a 100 μL aliquot of a LD₉₉ dose of a BoNT/A preparation and incubated for 60 minutes in a 22° C. water bath. Toxin dosing is based on the median weight per dose group, with mouse weights ranging from 17 grams to 30 grams, where the weight range of any single dose group of mice is no greater than +/−2 grams. The negative control is vehicle (0.5% BSA/saline solution) and the positive control is a hyperimmune rabbit serum containing a high titer of toxin-neutralizing antibodies. These test samples are then administered to the mice by intravenous injection in the tail (five mice/dose).

The next day, each mouse is prepared for thermal imaging under resting conditions as described above. A whole body thermal image of each mouse is then taken using, e.g., the thermographic imaging system described above, and the digital images are stored on a computer hard drive. Analysis of temperature differences between the first and the second thermal image for each test sample is performed, e.g., as described in Example 3. In addition, test mice are compared (qualitatively and quantitatively) to images from the positive control group (full protection; normothermic) and the negative control group (no protection; maximally hypothermic). Mice that do not exhibit a statistically significant increase in the thermal energy being emitted after administration of the test sample, i.e., protected from BoNT/A toxicity, as compared to control animals, e.g., the thermal images of negative control mice administered the LD₉₉ of the BoNT/A preparation, indicate that the patient has developed an immunoresistant response to the BoNT/A preparation (i.e. that toxin-neutralizing antibodies are present in the patient serum).

### Example 8

### Assessing a Physiological Activity of a Target Site for a Clostridial Toxin Administration Using Blood Flow

This example illustrates how examining blood flow can identify a target site for administering a Clostridial toxin.

A 44 year old male patient suffers from intense pain due to a task-specific dystonia affecting the palm and fingers of the right hand. To determine the location of the dystonic areas as well as to assess whether a Clostridial toxin administration would be appropriate for treating this affliction, the physician employs a laser-Doppler technique to assess the physiological activity of the man's palm and fingers of the right hand.

The male patient is prepared for laser-Doppler recording. The scanner unit is positioned at a distance of approximately 20 to 30 cm perpendicular to the affected hand, thereby allowing maximum coverage of the target site. Blood flow from the hand is then taken and the digital image is stored on a computer hard drive. One laser-Doppler system that may be used in accordance with aspects of the present invention is the laser-Doppler flowmeter (LDI, Moor Instruments, Ltd., Devon, UK). This device will scan the skin surface with a 2-mW helium laser across and registers the shifted frequency from the backscattered light. The velocity of moving red blood cells will be calculated and presented as a color-coded picture representing a relative measure of skin perfusion (laser-Doppler flux=velocity×concentration of moving red blood cells) in two dimensions. A target site of 7.5×7.5 cm² will be scanned repeatedly 4 times at a distance of approximately 25-35 cm from the skin. The image resolution obtained will be 118×70 pixels with a speed of 4 ms/px. Each single scan will last approximately 44 seconds. Scan intervals will be set at 45 seconds. Bandwidth will be set at 250 Hz to 15 Hz. Images will be analyzed using a dedicated image-processing software (Moor Instruments, Ltd., Devon, UK) Average blood flow in purfusion units will be obtained from baseline scanning and will be compared to average blood flow obtained at subsequent periods. After examination of the blood flow recordings, the physician determines that three muscle groups show a dramatically increase in blood being emitted from the affected hand as compared to the male patient's unaffected left hand and with blood flow recordings of hands that are taken from other patients unaffected by task-specific dystonia. The physician recommends administering a Clostridial toxin to the muscle groups showing increased blood flow to alleviate the task-specific dystonia.

### Example 9

### Administering a Clostridial Toxin to a Target Site Using Blood Flow

This example illustrates how examining blood flow will provide information regarding where to administer a Clostridial toxin to a target site.

A 38 year old female patient presents with a severe case of axillary hyperhidrosis. The treating physician recommends administering a botulinum toxin type A to the affected areas of hyperhidrosis. To determine which sweat glands to treat, the physician employs a thermal imaging technique to assess the physiological activity of the axillary areas undergoing excessive sweating.

The female patient is prepared for laser-Doppler recording as described in Example 8, except that the scanner unit is positioned at a distance of approximately 20-30 cm perpendicular to the affected axillary area in order to achieve maximum coverage of the target site and a target site of 20×20 cm² will be scanned. A laser-Doppler recording of the area is then taken as described in Example 8. After examination of the blood flow recordings, the physician determines reveals five target sites encompassing an 8×15 cm² region that exhibit a statistically significant increase in blood flow in the affected areas of hyperhidrosis in the female patient as compared to other patients unaffected by axillary hyperhidrosis.

The physician then proceeds to administer a Clostridial toxin to the areas showing increased blood flow to alleviate the excessive sweating. Based on the blood flow recordings, the target sites are mapped within the 8×15 cm² region. Crystal ice particle coated with Botulinum toxin type A are loaded into a needleless injector. The projection pressure is set so that the drug particles may be delivered to the dermis layer of the skin. Also, the amount of the drug particle is loaded so that approximately 20 units to approximately 60 units of botulinum toxin type A is delivered to the five target sites within the 8×15 cm² region.

### Example 10

### Assessing the Effect of a Clostridial Toxin Administration Using Blood Flow

This example illustrates how comparing blood flow recordings of a target site before and after the administration of a Clostridial toxin will provide information regarding the degree of a Clostridial toxin effect on that target site.

A 53 year old female patient suffers from intense pain due to a temporomandibular joint dysfunction. The treating physician recommends administering 10 units of botulinum toxin type A to her masseter muscles to alleviate the pain. To determine the muscle sites to treat, the physician employs a laser-Doppler technique to assess the physiological activity of the area undergoing intense pain.

The female patient is prepared for laser-Doppler recording as described in Example 8, except that the scanner unit is positioned at a distance of approximately 25-35 cm perpendicular to the affected temporomandibular joint area in order to achieve maximum coverage of the target site. In addition, all facial cosmetics are removed and the skin surface allowed to air dry. Hair is held back off the face with hair clips and a reflective marker is placed on the skin overlying the anterior edge of the masseter muscle. Blood flow recordings of both sides of the face are then taken as described in Example 8. After examination of the blood flow recordings, the physician determines that the masseter muscle on both sides of the face exhibit a statistically significant increase in the thermal energy being emitted from the affected experiencing pain in the female patient as compared to other patients unaffected by temporomandibular joint dysfunction.

The physician then proceeds to administer a Clostridial toxin to the areas showing increased blood flow to alleviate the pain. Based on the blood flow recordings, the target sites within the masseter muscle are mapped and the physician administers approximately 10 units of botulinum toxin type A to the target sites within the masseter muscles on each side of the patient's face of the patient. The patient is discharged and is asked to return for a second scan in 24 hours. Further, to prevent the unwanted dispersal of botulinum toxin into the adjacent muscles, the patient is instructed to not massage the administration site, and is advised to not reapply her makeup in the office.

The next day, the female patient returns and is prepared for a laser-Doppler recording as described above. Blood flow recordings of both sides of the face are then taken as described above. Comparison of the before-treatment and after-treatment blood flow recordings indicate that the muscles administered botulinum toxin show a decrease in blood flow which approximates the blood flow observed by masseter muscles from patients unaffected by temporomandibular joint dysfunction. This comparison also shows that muscles not administered botulinum toxin show a similar blood flow recording as patients unaffected by temporomandibular joint dysfunction. Upon analysis of these blood flow recordings, the physician determines that additional administration of botulinum toxin is not warranted.

### Example 11

### Assessing the Dispersal of a Clostridial Toxin Administration Using Blood Flow

This example illustrates how comparing blood flow recordings of a target site before and after the administration of a Clostridial toxin will provide information regarding the degree of a Clostridial toxin effect on a target site and any possible dispersal of the toxin away from the target site to a non-target site.

A 33 year old male patient suffers from intense pain due to a muscle spasm in his left calf. The treating physician recommends administering 10 units of botulinum toxin type A to the calf muscle to alleviate the pain. To make sure that the administered botulinum toxin does not diffuse to unintended muscles, the physician employs a laser-Doppler technique to assess the physiological activity of the area undergoing intense pain.

The male patient is prepared for laser-Doppler recording as described in Example 8, except that the scanner unit is positioned at a distance of approximately 45-55 cm perpendicular to the affected leg in order to achieve maximum coverage of both the target and non-target sites. Blood flow recordings of both sides of the face are then taken as described in Example 8. After examination of the blood flow recordings, the physician determines that three target sites that exhibit a statistically significant increase in blood flow from the spasmodic calf area of the male patient as compared to the unaffected right calf area of the male patient as well as other patients not experiencing muscle spasms in the calf.

The physician then proceeds to administer a Clostridial toxin to the areas showing increased blood flow to alleviate the muscle spasm and associated pain. Based on the blood flow recordings, the target sites within the calf muscles are mapped and the physician administers approximately 10 units of botulinum toxin type A to the target sites within the muscles of the left calf. The patient is discharged and is asked to return for a second scan in 24 hours. Further, to prevent the unwanted dispersal of botulinum toxin into the adjacent muscles, the patient is instructed to not massage the target site and avoid exertion on the day of treatment.

The next day, the male patient returns and is prepared for a laser-Doppler recording as described above. Blood flow recordings of the left and right calves are then taken as described above. Comparison of the two blood flow recordings indicate that most of the calf muscles administered botulinum toxin show a decrease in blood flow which approximates the blood flow observed by the unaffected right calf muscles from the patient. However, a small region from one of the identified target sites still emits an increased blood flow, indicating an additional toxin administration is required. In addition, examination of the non-target sites reveal that these sites do not show a change in blood flow, indicating that the toxin did not diffuse into these non-target sites. Therefore, upon analysis of these blood flow recordings, the physician determines that additional administration of botulinum toxin should be administered in the remaining target site showing a difference in blood flow relative to the unaffected right calf muscle.

### Example 12

### Assessing the Effective Threshold Toxic Dose of a Clostridial Toxin Administration Using a Systemic Blood Flow Assay

This example illustrates how the effective threshold dose of a Clostridial toxin formulation can be determined by assessing the effect of a Clostridial toxin administration in a mammal using differences in blood flow.

Currently, the effective lethal dose of a Clostridial toxin is determined using an in vivo assay that measures animal lethality, such as, e.g., the mouse lethality assay (MLA or LD₅₀ assay). The standard LD₅₀ assay evaluates the dose-dependent lethality of toxin preparations. However, the high doses of a Clostridial toxin necessary to achieve lethality also result in a systemic hypothermic response in the animal due to the disruption of many physiological processes that effect blood flow. This induced blood flow response, due to the systemic responses to a Clostridial toxin administration, can be used as a readout of a toxin effect. In addition, Clostridial toxin-mediated changes in the physiological state of a mammal occur well before the onset of lethality. Thus, the detectable blood flow changes resulting from the milder toxicity of lower non-lethal doses of a toxin can be used as an assay endpoint to determine the threshold systemic effects of a toxin rather than the lethal effects of the toxin. Therefore determining the effective threshold dose of a Clostridial toxin using a blood flow assay will greatly reduce the pain and suffering of the animals.

To determine the effective threshold dose of a Clostridial toxin formulation, the effect of a Clostridial toxin administration is assessed using a blood flow assay. Mice are prepared for laser-Doppler recordings. Mice are then lightly anesthetized with Isoflurane and then prepared for laser-Doppler recording as described in Example 8, except that the scanner unit is positioned at a distance of approximately 10-20 cm perpendicular to the skin surface of the ventral thorax region of the animal. Various doses of a BoNT/A formulation are then administered to the mice following recovery from anesthesia. In a typical assay, a BoNT/A stock solution is used to generate dose dilutions over a dose range of 30-60 U/kg (e.g. 30 U/kg, 36 U/kg, 42 U/kg, 48 U/kg, 54 U/kg, and 60 U/kg), with dilutions made in vehicle (0.5% BSA/saline solution). Dosing is based on the median weight per dose group, with mouse weights ranging from 17 grams to 30 grams, where the weight range of any single dose group of mice is no greater than +/−2 grams, and dose groups consist of 10 mice each. Mice are injected intraperitoneally with either vehicle (control) or the specific toxin dose dilution, delivered via a 27 gauge needle, and each mouse receives a dose volume based on individual weight, such that the desired dose (in U/kg) is delivered in a volume of 10 mL/kg.

The next day, each mouse is prepared for laser-Doppler recordings as described above. Analysis of blood flow differences between the first and the second recordings are performed and a dose response curve is derived from nonlinear regression analysis of these data, establishing a blood flow dose-response measure of non-lethal toxicity. The dose that results in 50% of the mice exhibiting a statistically significant decrease in blood flow after administration of the BoNT/A preparation as compared to control animals, e.g., blood flow of the same mice before administration of the BoNT/A preparation or different mice administered a saline control (a normothermic vehicle control group), is the effective threshold dose (TD₅₀).

### Example 13

### Assessing the Effective Threshold Pharmacological Dose of a Clostridial Toxin Administration Using a Local Target Site Blood Flow Assay

This example illustrates how the effective threshold dose of a Clostridial toxin formulation can be determined by assessing the effect of a Clostridial toxin administration in a mammal using differences in blood flow.

The effective pharmacological dose of a Clostridial toxin is determined using an in vivo assay that measures muscle paralysis, such as, e.g., the Digit Abduction Score (DAS) assay or the Gastrocnemius Paralysis Assay (GPA). Muscle paralysis results in a decrease in blood flow by an exercised muscle due to failure in muscle contraction. This decrease in induced blood flow following exercise (hypothermia) can be used as a readout of a Clostridial toxin effect. The doses used in pharmacological studies are non-toxic and non lethal and should only elicit paralytic responses in the muscle of injection (e.g. gastrocnemius) or in adjacent muscles as a measure of local intermuscular diffusion (e.g. tibialis anterior, extensor digitorum longus, quadriceps).

To determine the effective pharmacological dose of a Clostridial toxin formulation, the effect of a Clostridial toxin administration is assessed using a blood flow assay. Mice are prepared for a laser-Doppler recording under non-resting conditions by physical stimulation using a treadmill (15 degree incline @ 5 meters/minute) for 10 minutes. Mice are then lightly anesthetized with Isoflurane and then prepared for laser-Doppler recording as described in Example 8, except that the scanner unit is positioned at a distance of approximately 10-20 cm perpendicular to the skin surface (fur shaved) overlying the right (ipsilateral) and left (contralateral) gastroxcnemius muscles (fur shaved) of each mouse is recorded. Various doses of a BoNT/A formulation are administered to the mice by intramuscular injection in the tail (five mice/dose). In a typical assay, a BoNT/A stock solution is used to generate dose dilutions over a dose range of 1-60 U/kg, with dilutions made in vehicle (0.5% BSA/saline solution). Dosing is based on the median weight per dose group, with mouse weights ranging from 17 grams to 30 grams, where the weight range of any single dose group of mice is no greater than +/−4 grams, and dose groups consist of 6 mice each. The injection volume is 5 or 10 μL, delivered via a 30 gauge needle using a Hamilton syringe affixed with a ratcheting, volume-adjustable dispenser. BoNT/A solution is injected into the distal portion of the medial head of the right gastrocnemeus muscle, using the posterior medial malleolar groove as a needle guide. Progressive paralysis is then evaluated daily for four days to capture the peak paralytic effects (typically between three to four days post-injection of BoNT/A). Prior to collection of blood flow recordings, mice are similarly exercised on the treadmill to stimulate muscle activity, followed by anesthesia and recording. Blood flow recordings from test groups are compared (qualitatively and quantitatively) to blood flow recordings from the vehicle control group (normothermic). The degree of blood flow difference (net difference from vehicle control) is calculated for each dose administered and an ED₅₀ (the dose producing 50% paralysis) is derived from nonlinear regression analysis of these data, establishing a blood flow dose-response measure of muscle paralysis.

### Example 14

### Assessing Immunoresistance to a Clostridial Toxin Administration Using a Blood Flow Assay

This example illustrates how immunoresistance to a Clostridial toxin can be determined by assessing the effect of a Clostridial toxin administration in a mammal using differences in blood flow.

Immunoresistance to a Clostridial toxin in a mammal is usually determined using an in vivo assay that measures animal lethality, such as, e.g., the mouse protection assay (MPA). The current standard MPA evaluates the degree of protection conferred by anti-Clostridial toxin-neutralizing antibodies against a lethal challenge dose of a toxin. However, the high doses of a Clostridial toxin necessary to achieve lethality also result in a systemic blood flow response in the animal due to the disruption of many physiological processes that effect blood flow. This induced blood flow response, due to the systemic responses to a Clostridial toxin administration, can be used as a readout of a toxin effect. In addition, Clostridial toxin-mediated changes in the physiological state of a mammal occur well before the onset of lethality. Thus, the detectable blood flow changes resulting from the milder toxicity of lower non-lethal doses of a toxin can be used to infer the presence of toxin-neutralizing antibodies since the presence of toxin-neutralizing antibodies will effectively lower the challenge toxin dose (when combined), producing a graded toxicity response to the otherwise lethal challenge dose. Therefore determining the presence of anti-Clostridial toxin-neutralizing antibodies using a blood flow assay will greatly reduce the pain and suffering of the animals.

To determine the immunoresistance of a cervical dystonia patient, the effect of a Clostridial toxin administration is assessed using a blood flow assay. First, the maximum toxic dose (LD₉₉) of a BoNT/A preparation is determined, e.g., as described in Example 12, except that the various doses of a BoNT/A formulation are administered to the mice by intravenous injection in the tail. Second, mice are prepared for a laser-Doppler recording under resting conditions. Mice are then lightly anesthetized with Isoflurane and then prepared for laser-Doppler recording as described in Example 8, except that the scanner unit is positioned at a distance of approximately 10-20 cm perpendicular to the skin surface of the ventral thorax region of the animal. A blood sample from each patient is processed to obtain the serum. A 100 μL aliquot of serum from each patient is mixed with a 100 μL aliquot of a LD₉₉ dose of a BoNT/A preparation and incubated for 60 minutes in a 22° C. water bath. Toxin dosing is based on the median weight per dose group, with mouse weights ranging from 17 grams to 30 grams, where the weight range of any single dose group of mice is no greater than +/−2 grams. The negative control is vehicle (0.5% BSA/saline solution) and the positive control is a hyperimmune rabbit serum containing a high titer of toxin-neutralizing antibodies. These test samples are then administered to the mice by intravenous injection in the tail (five mice/dose).

The next day, each mouse is prepared for laser-Doppler recordings as described above. Analysis of blood flow differences between the first and the second blood flow recordings is performed. In addition, test mice are compared (qualitatively and quantitatively) to blood flow recordings from the positive control group (full protection; normothermic) and the negative control group (no protection; maximally hypothermic). Mice that do not exhibit a statistically significant increase in blood flow after administration of the test sample, i.e., protected from BoNT/A toxicity, as compared to control animals, e.g., the blood flow recordings of negative control mice administered the LD₉₉ of the BoNT/A preparation, indicate that the patient has developed an immunoresistant response to the BoNT/A preparation (i.e. that toxin-neutralizing antibodies are present in the patient serum).

### Example 15

### Evaluation of the Effects of Botulinum Toxin Pretreatment on The Peripheral Blood Flow Response Due to Acute Subcutaneous Capsaicin Treatment, in the Plantar Surface of the Rat Hindpaw

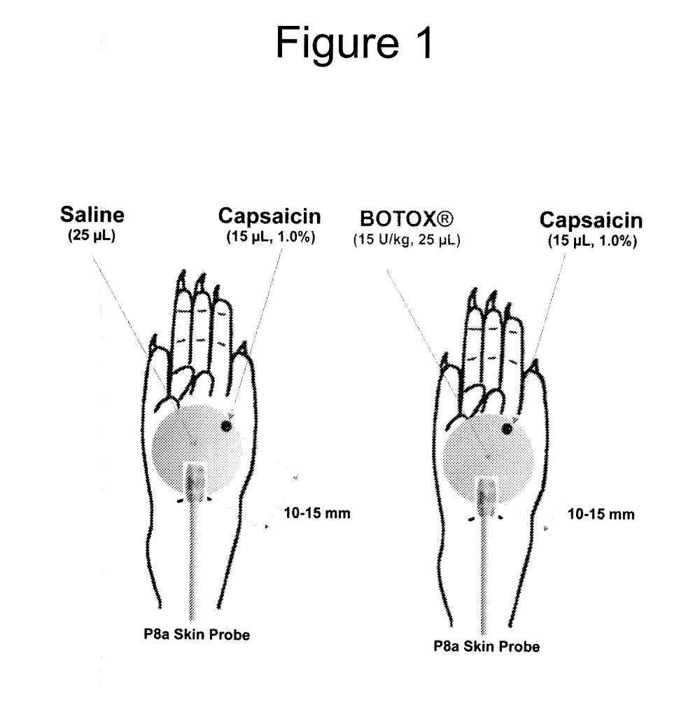

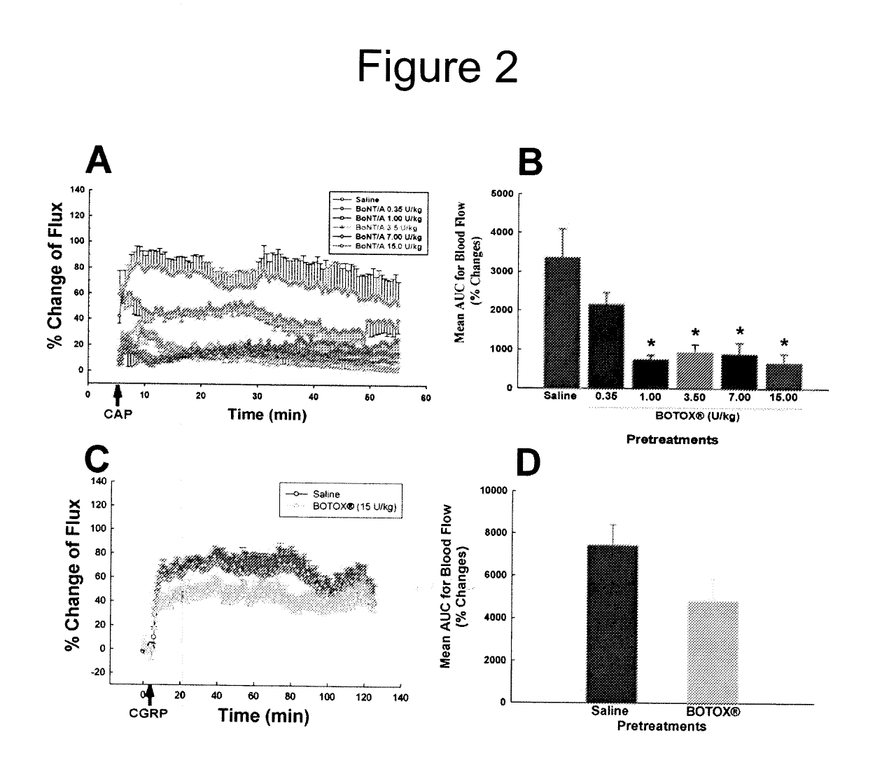

Botulinum toxin type A (BoNT/A) was injected subcutaneously into the plantar surface of the rat right hindpaw using a Hamilton syringe fitted with a 25 gauge needle (FIG. 1). A 100 unit vial of BoNT/A was reconstituted in 500 uL of normal saline, yielding a concentration of 0.2 units/uL. For rats averaging 330 g (i.e., approximately one third of a kilogram), a maximally efficacious dose of 15 units/kg (FIG. 1 & FIG. 2) was delivered by injecting 25 uL of the reconstituted BoNT/A solution. As a control for the toxin effects, a separate set of rats were injected similarly with 25 uL of saline only. BoNT/A was also serially diluted in saline to yield lower doses (3.5 units/kg, 7 units/kg), to assess the dose-dependency of the response. Rats were then tested for the pretreatment effects of BoNT/A at various time points (e.g., 1 day post treatment, 3 days, 7 days, 14 days, 28 days) in the acute capsaicin model.

In this example, peripheral blood flow was evaluated in the plantar surface of the right hindpaw in anesthetized rats. Capsaicin (Sigma, St. Louis, Mo.) was prepared as a 1% solution in a vehicle containing saline (93%) and Tween 80 (7%) (v/v). A volume of 15 ul of this 1% capsaicin solution was injected into the plantar surface of the hindpaw, such that the capsaicin injection overlapped with the area pretreated with BoNT/A (FIG. 1). The rat was maintained on a feedback-regulated thermal pad, to stabilize core temperature, and anesthesia and ventilation were maintained through a nose cone mask.

Capsaicin-induced vasodilatation was measured by recording the changes in subcutaneous blood flow in the plantar surface of the right hindpaw using a dual laser Doppler flow meter device (moorLAB DRT4, Moor Instruments Ltd, Axminster, Devon, UK). Probes were placed on the rat hind paw in light contact with the plantar skin (FIG. 1), using adhesive tape, and avoiding any skin compression that might introduce experimental artifacts. The analog output signals from the laser Doppler were captured by using CED 1401 with Spike2 software. Spike2 was also used for off-line data analysis. Raw data was captured at 2.5 kHz and filtered down to one point per 30 seconds. Baseline recordings were captured for 5 minutes duration prior to capsaicin injection. All blood flow values were compared with the averaged baseline to calculate the percent change of blood flow (flux) before and after capsaicin injection (FIG. 2).

Acute subcutaneous capsaicin treatment produced a significant increase in peripheral blood flow, as measured by laser Doppler, lasting greater than 60-90 minutes post injection (FIGS. 2A & 2C). Pretreatment with BoNT/A significantly reduced this observed increase in blood flow in a dose-dependent manner (FIG. 2A). A maximally efficacious subcutaneous dose of 15 units/kg, in a 25 uL injection volume, reduced blood flow back to near baseline levels (FIG. 2A). In contrast, direct application of the vasodilating agent, CGRP, produced an increase in blood flow that was not inhibited by BoNT/A pretreatment (FIGS. 2C & 2D). These results support an inhibitory effect of Botulinum toxin on neurogenically-mediated changes in blood flow.

### Example 16

### Monitoring the Development of Peripheral Neuropathy by Evaluating Peripheral Blood Flow Responses in the Plantar Surface of The Rat Hindpaw, in the Streptozotocin (STZ)-Induced Peripheral Diabetic Neuropathic Pain Model

Diabetes was induced in Sprague-Dawley male rat (Charles River) weighing 240-280 g by a single injection of STZ (Sigma, St. Louis, Mo.). STZ was freshly dissolved in citrate buffer (pH 4.5) to a concentration of 65 mg/ml and injected through the rat tail vein (65 mg/kg at 1 mL/kg, intravenously). Control rats received an equal volume of vehicle (saline or citrate buffer). To monitor the onset of diabetes, blood samples were collected and tested. Rats were warmed under a heat lamp to visualize the lateral tail vein. While being gently held in an acrylic restraining box, with the tail free, a 25 gauge needle was inserted (bevel up) into the lateral tail vein and blood was collected into heparinized containers. Blood levels of glucose, cholesterol (total cholesterol and HDL) and triglycerides were monitored using a CardioChek PA Analyzer (Polymer Technology Systems, Inc., Indianapolis, Ind.). A consequence of the STZ model is the progressive degeneration and dysfunction of the peripheral nervous system. This results in the tandem observation of both the progressive development of pain (peripheral neuropathic pain) and the progressive loss of neurogenic vasodilatation.

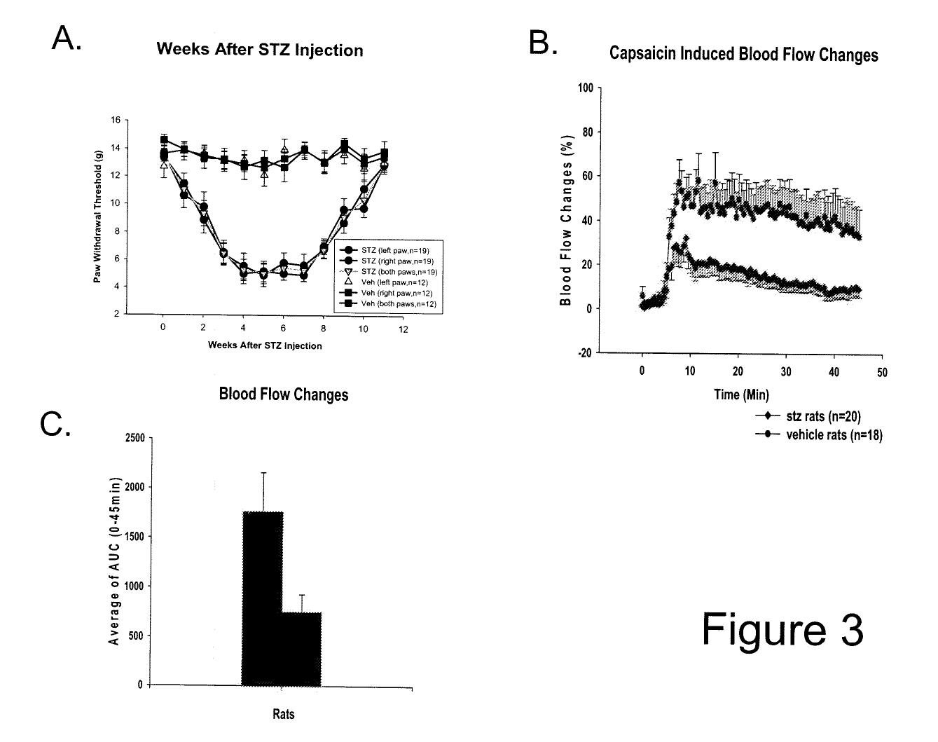

To evaluate the progressive development of peripheral neuropathic pain, mechanical allodynia was assessed weekly (post STZ-injection) by determining the paw withdrawal threshold (PWT) (FIG. 3A). A set of von Frey filaments (bending force from 1 g-15 g) were used to determine the 50% PWT in rats by using the up-down method of Dixon, see, e.g., W. J. Dixon, Efficient Analysis of Experimental Observations, 20 Annu. Rev. Pharmacol. Toxicol. 441-462 (1980); and S. R. Chaplan et al., Quantitative Assessment of Tactile Allodynia in the Rat Paw, 53(1) J. Neurosci. Methods 55-63 (1994). Mechanical allodynia (peripheral neuropathic pain) was deemed to be established when a rat displayed two consecutive 50% PWT values below 6 g on the von Frey scale, separated by at least 3 days (compared to 15 g for native rats) (FIG. 3A). Typically, a stable level of allodynia was established in 3-4 weeks post STZ treatment and maintained for an additional 3-4 weeks. Eventually, though, the progressive neurodeneration and dysfunction results in a spontaneous reversal of the allodynia back to baseline levels of response, indicating a complete desensitization to peripheral mechanical stimulation (FIG. 3A).

In parallel to the assessment of the peripheral neuropathic pain (allodynia), peripheral neurogenic vasodilatation was assessed weekly over the entire time course of the allodynia assessments as a secondary measure of peripheral nerve function, using acute subcutaneous administration of capsaicin. Since capsaicin itself can produce a reversible desensitization, neurogenic vasodilatation was only determined once in each rat (control or test subjects), and each weekly test was done in a separate group of rats aged to the appropriate time point. Rats were anesthetized prior to blood flow evaluation. Each test subject was maintained on a feedback-regulated thermal pad, to stabilize core temperature, and anesthesia and ventilation were maintained through a nose cone mask. Capsaicin-induced vasodilatation was measured by recording the changes in subcutaneous blood flow in the plantar surface of the right hindpaw using a dual laser Doppler flow meter device (moorLAB DRT4, Moor Instruments Ltd, Axminster, Devon, UK). Probes were placed on the rat hind paw in light contact with the plantar skin, using adhesive tape, and avoiding any skin compression that might introduce experimental artifacts (FIG. 1). The analog output signals from the laser Doppler were captured by using CED 1401 with Spike2 software. Spike2 was also used for off-line data analysis. Raw data was captured at 2.5 kHz and filtered down to one point per 30 seconds. Baseline recordings were captured for 5 minutes duration prior to capsaicin injection. All blood flow values were compared with the averaged baseline to calculate the percent change of blood flow before and after capsaicin injection.

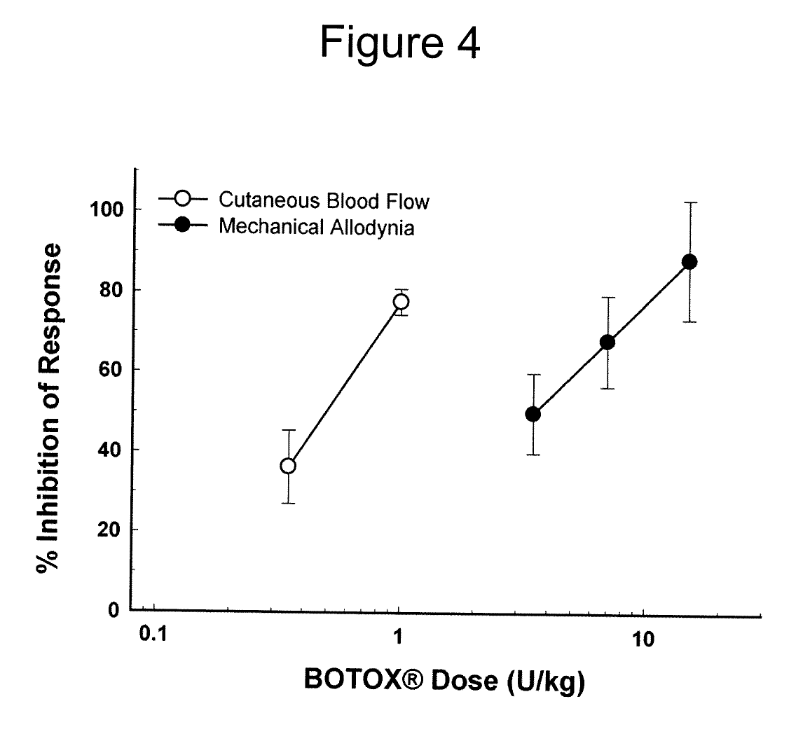

The observed capsaicin-induced increase in peripheral blood flow (neurogenic vasodilatation) was observed to decrease over the time course of the established allodynia. At the point of pain desensitization (spontaneous reversal of the allodynia response back to the baseline), the capsaicin-induced neurogenic vasodilatation was significantly decreased compared to control rats (FIGS. 3B & 3C). This suggests a dysfunction of the peripheral nervous system mediating the neurogenic vasodilatation response. Additionally, the dose responses for inhibition of blood flow compared to inhibition of mechanical allodynia were parallel (FIG. 4). However, inhibition of blood flow was sensitive to much lower doses of BoNT/A than inhibition of mechanical allodynia (pain). Thus, this supports the use of peripheral blood flow measurement as a marker of peripheral nerve function and as a surrogate readout for concomitant pain states due to neurogenic inflammation.

Although the invention has been described with reference to the examples provided above, it should be understood that various modifications can be made without departing from the spirit of the invention. Accordingly, the invention is limited only by the following claims.

What is claimed:

1. A method of assessing an effect of a Clostridial toxin on a target site in a mammal, the method comprising the steps of:
a. recording a first blood flow from a surface of the target site in the mammal prior to administration of the Clostridial toxin;
b. recording a second blood flow from the surface of the target site in the mammal after the administration of the Clostridial toxin; and
c. comparing the first blood flow recording of step (a) to the second blood flow recording of step (b).
wherein a difference between the first blood flow recording and the second blood flow recording is indicative of a Clostridial toxin effect.

2. A method of assessing dispersal of a Clostridial toxin from a target site to a non-target site in a mammal, the method comprising the steps of:
a. recording a first blood flow from a surface of the target site in the mammal and a first blood flow from a surface of the non-target site of the mammal prior to administration of the Clostridial toxin;
b. recording a second blood flow from the surface of the target site in the mammal and a second blood flow from the surface of the non-target site of the mammal after the administration of the Clostridial toxin; and
c. comparing the first blood flow recording of the target site and the first blood flow recording of the non-target site of step (a) to the second blood flow recording of the target site and the second blood flow recording of the non-target site of step (b);
wherein a difference between the first blood flow recording of the non-target site and the second blood flow recording of the non-target site is indicative of dispersal of a Clostridial toxin from a target site.

3. A method of assessing an inhibitory effect of a Clostridial toxin on neurogenic inflammation in a target site of a mammal, the method comprising the steps of:
a) administering to a mammal an effective amount of a Clostridial toxin to a target site and a control treatment to a non-target site;
b) administering an effective amount of a challenger to the target site and to the non-target site, wherein the challenger is administered after the administration of the Clostridial toxin; and
c) recording the blood flow in the target site and non-target site;
wherein a lower blood flow in the target site as compared to the non-target site is indicative of a Clostridial toxin effect on neurogenic inflammation.

4. The method according to claim 3, wherein the challenger is an Aα-fiber agonist, an Aβ-fiber agonist, an Aγ-fiber agonist, an Aδ-fiber agonist, or a C-fiber agonist.

5. A method of assessing an inhibitory effect of a Clostridial toxin on pain in a target site of a mammal, the method comprising the steps of:
a) administering to a mammal an effective amount of a Clostridial toxin to a target site and a control treatment to a non-target site;
b) administering an effective amount of a challenger to the target site and to the non-target site, wherein the challenger is administered after the administration of the Clostridial toxin; and
c) recording the blood flow in the target site and non-target site;
wherein a lower blood flow in the target site as compared to the non-target site is indicative of a Clostridial toxin effect on pain.

6. The method according to claim 3, wherein the challenger is an Aα-fiber agonist, an Aβ-fiber agonist, an Aγ-fiber agonist, an Aδ-fiber agonist, or a C-fiber agonist.
